(19) United States
(12) Patent Application Publication
(10) Pub. No.: US 2022/0152167 A1
(43) Pub. Date: May 19, 2022

(54) IMMUNOGENIC FORMULATIONS FOR TREATING CANCER

(71) Applicant: UNIVERSIDAD DE CHILE, Santiago (CL)

(72) Inventors: Flavio Andres SALAZAR ONFRAY, Santiago (CL); Cristian Javier PEREDA RAMOS, Santiago (CL); Mercedes Natalia LOPEZ NITSCHE, Santiago (CL); Maria Alejandra GLEISNER MUNOZ, Santiago (CL); Andres TITTARELLI, Santiago (CL); Fermin GONZALEZ, Santiago (CL); Fabian TEMPIO, Santiago (CL); Marisol BRIONES, Santiago (CL)

(21) Appl. No.: 17/436,380

(22) PCT Filed: Mar. 5, 2020
(86) PCT No.: PCT/IB2020/051906

§ 371 Date: Sep. 3, 2021

Related U.S. Application Data
(60) Provisional application No. 62/814,756, filed on Mar. 6, 2019.

(51) Int. Cl. A61K 39/00; A61P 35/00

(52) U.S. Cl. CPC A61K 39/0011; A61P 35/00; A61K 2039/876; A61K 2039/55516; A61K 2039/572; A61K 2039/5154

(57) ABSTRACT

Aspects of the present disclosure generally relate to immunotherapy, cancer vaccines and the treatment of cancer diseases. By way of example, the present disclosure relates to novel combined with an immunologically effective amount of adjuvant, for treating cancer in a subject methods of generating such formulations, and methods of use thereof.

### CROSS-REFERENCE TO RELATED APPLICATION

This application claims the benefit of U.S. Provisional Application No. 62/814,756, filed Mar. 6, 2019, which is hereby incorporated by reference in its entirety.

### FIELD OF THE INVENTION

The present invention relates generally to immunotherapy, cancer vaccines and the treatment of cancer diseases. In particular, it relates to novel immunogenic formulations of cell lysates from heat-shock conditioned tumor cell populations combined with an immunologically effective amount of adjuvant, for treating cancer in a subject and methods thereof.

### BACKGROUND OF THE INVENTION

Immunotherapy based on immune-checkpoint blockers has proven survival benefits in patients with melanoma and other malignancies (Larkin, et al. 2015. Combined nivolumab and ipilimumab or monotherapy in untreated melanoma. N Engl J Med 373:23-34). Nevertheless, a significant proportion of treated patients remain refractory, suggesting that combinations with active immunizations, such as cancer vaccines, could be helpful to improve response rates.

In this context, cancer vaccines, particularly tumor-based vaccines and the use of dendritic cells (DCs), resurge as an alternative for complementary immunological treatments in cancer patients. Here, optimal delivery of a wide-ranging pool of Tumor-Associated Antigens (TAAs) and the use of adequate adjuvants are shown to be crucial for vaccine success (Andrews et al. 2008. Cancer vaccines for established cancer: how to make them better? Immunol Rev 222:242-255).

For instance, the U.S. Pat. No. 9,694,059 discloses an ex vivo process to obtain activated antigen-presenting cells (APCs) useful for treating cancer and immune system-related diseases. This DCs-like APCs are generated through its in vitro activation with heat-shock conditioned tumor cell lysates (CTCL), particularly derived from melanoma cell lines. Sixty percent of advanced melanoma patients treated with these APCs showed a delayed type hypersensitivity reaction against antigens contained in the CTCL, which correlated with a three-fold prolonged patient survival (López et al. 2009. Prolonged survival of dendritic cell-vaccinated melanoma patients correlates with tumor-specific delayed type IV hypersensitivity response and reduction of tumor growth factor beta-expressing T cells. J Clin Oncol 27:945-952) (Aguilera et al. 2011. Heat-shock induction of tumor-derived danger signals mediates rapid monocyte differentiation into clinically effective dendritic cells. Clin Cancer Res 17:2474-2483).

Despite these positive results, two problems limit the transfer of CTCL-activated antigen presenting cell-based therapy. The first, of biological nature, is revealed by the fact that a high percentage of patients (40%) do not respond to the therapy, which could be explained in part by biological differences between patient's tumors (Hélias-Rodzewicz et al. 2015. Variations of BRAF mutant allele percentage in melanomas. BMC Cancer 15: 497); the state of the patient's immune system-tumor relationship (Durán-Aniotz et al. 2013. The immunological response and post-treatment survival of DC-vaccinated melanoma patients are associated with increased Th1/Th17 and reduced Th3 cytokine responses. Cancer Immunol Immunother 62:761-772); genetic differences in components of the immune system of patients (Tittarelli et al. 2012. Toll-like receptor 4 gene polymorphism influences dendritic cell in vitro function and clinical outcomes in vaccinated melanoma patients. Cancer Immunol Immunother 61:2067-2077) (Garcia-Salum et al. 2018. Molecular signatures associated with tumor-specific immune response in melanoma patients treated with dendritic cell-based immunotherapy. Oncotarget 9:17014-17027); or on the other hand by deficiencies in the processing and presentation of antigens by the injected APCs (Cynthia M. Fehres et al. 2014. Understanding the Biology of Antigen Cross-Presentation for the Design of Vaccines Against Cancer. Front Immunol. 5: 149). An additional difficulty is of technological nature. The therapy based on APCs and/or DC vaccines requires an infrastructure and a highly specialized team, which generates elevated production costs and makes the business model difficult by requiring a personalized production and therefore affects the technology transfer, which has been reflected in the experience of Dendreon (Ledford 2015. Therapeutic cancer vaccine survives biotech bust. Nature 519:17-18).

A promising alternative corresponds to the targeting in vivo of DCs via therapeutic vaccines based on whole tumor cell lysates, or tumor-derived compounds (such as mRNA, recombinant tumor proteins, or antigenic peptides) as direct source of antigens (Guo et al. 2013. Therapeutic cancer vaccines: past, present, and future. Adv Cancer Res 119:421-475). Although many studies on anti-tumor vaccines use tumor lysates ex vivo-treated DCs, we can find little research using whole tumor lysates to immunize patients directly. In general, the effects of these whole tumor cell-based vaccines (both ex vivo loaded DC vaccines or direct immunization with tumor cells) have been limited and, in most cases, without achieving growth retardation or regression of tumor size (Sondak & Sosman 2003. Results of clinical trials with an allogeneic melanoma tumor cell lysate vaccine: Melacine. Semin Cancer Biol 13:409-415) (Melief et al. 2015. Therapeutic cancer vaccines. J Clin Invest 125:3401-3412) Between the years 1987 and 2000, several clinical studies were conducted in order to test the effectiveness of a vaccine based on the use of a lysate obtained from two tumor cell lines plus an adjuvant against melanoma (Sondak & Sosman. 2003. Results of clinical trials with an allogeneic melanoma tumor cell lysate vaccine: Melacine. Semin Cancer Biol 13:409-415). The results showed that the therapy was not more effective than the traditional treatments, probably due to the lack of factors promoting the maturation of the APCs and the required induction of local inflammation.

In a recent randomized phase II trial in metastatic melanoma patients, the clinical effectiveness of autologous DC vaccines loaded ex vivo with autologous irradiated tumor cell cultures was compared to the effect of autologous irradiated tumor cell vaccines. The DC vaccine arm was associated with a doubling of median overall survival compared with the whole irradiated tumor vaccine (43.4 vs. 20.5 months) (Dillman et al. 2018. Randomized phase II trial of autologous dendritic cell vaccines versus autologous tumor cell vaccines in metastatic melanoma: 5-year follow up and additional analyses. J Immuno Ther Cancer 6:19). The median survival of 20.5 months in the tumor cell vaccine arm suggested that this kind of vaccines might also have anti-tumor activity. However, in this study no tumor regression was observed. Additionally, this strategy is only feasible in patients with resectable tumor and depends of the success in establishing autologous tumor cell lines.

In a large phase III study of postsurgical adjuvant therapy involving 496 stage IV melanoma patients, the clinical efficacy of an allogeneic whole-cell vaccine (Canvaxin, comprised of three irradiated whole cells melanoma lines: M10-VACC, M24-VACC, and M101-VACC) plus BCG was compared with BCG/placebo. The results showed that BCG/Canvaxin did not improve outcomes over BCG/placebo (Faries et al. 2017. Long-Term Survival after Complete Surgical Resection and Adjuvant Immunotherapy for Distant Melanoma Metastases. Ann Surg Oncol 24:3991-4000).

Recent studies have shown the importance of tumor cell danger signals release for enhancing adjuvant activity of tumor cells used as cancer vaccines. An example of that is the immunogenic cell death (the so-called necroptosis, or “programmed necrosis”), which when induced in tumor cells makes them immunogenic, both in vitro and in vivo, and when used as a vaccine they are capable of generating a powerful antitumor immune response (Aaes et al. 2016. Vaccination with Necroptotic Cancer Cells Induces Efficient Anti-tumor Immunity. Cell Rep 15:274-287).

In a series of in vitro studies, we have demonstrated that heat-shock conditioned melanoma cells are able to induce a variety of stress signals, such as release of Heat Shock Proteins (HSPs), the ATP release, the translocation of calreticulin (CRT, a well described “eat-me” signal), and the release of the chromatin-associated protein high-mobility group box 1 (HMGB1) that can act as an adjuvants in Ag delivery. These signals are closely related to the immunogenicity of tumor cell lysates, promoting APC maturation and enhancing antigen cross-presentation (Aguilera et al. 2011. Heat-shock induction of tumor-derived danger signals mediates rapid monocyte differentiation into clinically effective dendritic cells. Clin Cancer Res 17:2474-2483) (Gonzalez F. et al. 2014. Tumor cell lysates as immunogenic sources for cancer vaccine design. Human Vaccines & Immunotherapeutics 10:11, 3261-3269). Therefore, the induction of danger signals from the tumor cells prior to the lysis and irradiation steps together with the use of strong immune stimulant adjuvants could surpass the low clinical efficacy of whole-tumor cell vaccines.

Hemocyanins are enormous oligomers with a basic structure of a decamer composed of 10 subunits, ranging from 350 to 550 KDa, that are self-assembled into a cylinder of approximately 35 nm in diameter and 18 nm in height (Markl 2013. Evolution of molluscan hemocyanin structures. Biochim Biophys Acta 183:1840e1852). In the hemocyanins of gastropods, such as Concholepas Concholepas Hemocyanin (CCH), Fissurella latimarginata hemocyanin (FLH) and keyhole limpet hemocyanin (KLH), the decamers are assembled in pairs forming mostly didecamers. Hemocyanins have the ability to bias the immune response towards a Th1 phenotype (Becker et al. 2014. Mollusk hemocyanins as natural immunostimulants in biomedical applications. G. H. T. Duc (Ed.), Immune Response Activation, InTech, Rijeka, Croatia: pp. 45-72), activating the immune system which breaks the state of equilibrium in which cancer cells resist immune-mediated cell death. The use of CCH and FLH during anti-cancer therapy for recurrent superficial bladder cancer after transurethral surgical resection has been reported with negligible toxic side effects, making them ideal for long-term ongoing treatments (Arancibia et al. 2012. Hemocyanins in the immunotherapy of superficial bladder cancer. A. Canda (Ed.), Bladder Cancer from Basic to Robotic Surgery, INTECH, Croatia: 221-242). Concerning to its immunological properties, FLH is highly immunogenic and has been shown to be a better antitumor agent in a melanoma model than CCH or KLH (Arancibia et al. 2014. A novel immunomodulatory hemocyanin from the limpet Fissurella latimarginata promotes potent anti-tumor activity in melanoma. PLoS One 9:e87240). Currently, CCH is used as an adjuvant in a vaccine based on DCs loaded with prostate tumor cell lysates, which has been shown to be safe and effective to induce the T cell memory response in prostate cancer patients (Reyes et al. 2013. Tumour cell lysate-loaded dendritic cell vaccine induces biochemical and memory immune response in castration-resistant prostate cancer patients. Br J Cancer 109:1488-1497). Regarding the mechanisms of action of these large glycoproteins, CCH, FLH and KLH are internalized by the APCs through the participation of C-type lectin receptors such as mannose receptors (Presicce et al. 2008. Keyhole limpet hemocyanin induces the activation and maturation of human dendritic cells through the involvement of mannose receptor. Mol Immunol 45:1136-1145; Zhong et al. 2016. Hemocyanin stimulates innate immunity by inducing different temporal patterns of proinflammatory cytokine expression in macrophages. J Immunol 196:4650-4662).

In the patent U.S. Pat. 9,694,059 a heat-shock conditioned tumor cell lysate is used for the ex vivo stimulation of peripheral blood monocytes pre-activated with granulocyte macrophage colony stimulating factor (GM-CSF) and interleukin-4 (IL-4) in order to produce the differentiation, maturation and antigen loading of DCs. In the production of those Tumor Antigen Presenting Cells (TAPCells), the cell lysate fulfills a dual role, serving as a source of TAAs and acting as an activation factor through the cells' danger signals.

Canvaxin™ is an allogeneic whole-cell vaccine for melanoma comprised of three irradiated whole-cell melanoma lines suspended in culture medium containing human serum albumin and dimethyl sulfoxide. Another allogeneic whole-cell vaccine against melanoma comprised 5×10⁶ cells of three melanoma cell lines (IIB-MEL-J, IIB-MEL-LES, IIB-MEL-IAN) exponentially growing and irradiated with 5000 cGy and frozen in liquid nitrogen in medium containing 20% fetal bovine serum −10% DMSO (Mordoh et al. 1997. Allogeneic cells vaccine increases disease-free survival in stage III melanoma patients. A non randomized phase II study. Medicina (B Aires) 57:421-427). The same group developed another whole tumor cell vaccine against melanoma (CSF-470 Vaccine or Vaccimel), which consists of 1.6×10⁷ lethally irradiated cells derived from four cutaneous melanoma cell lines established in-house, MEL-XY1, MEL-XY2, MEL-XY3, and MEL-XX4. For CSF-470 vaccine preparation, the four cell lines are thawed, washed, mixed, and subsequently irradiated at 70 Gy. The vaccine is coadjuvated with BCG and recombinant rhGM-CSF (Mordoh et al. 2017. Phase II Study of Adjuvant Immunotherapy with the CSF-470 Vaccine Plus Bacillus Calmette-Guerin Plus Recombinant Human Granulocyte Macrophage-Colony Stimulating Factor vs. Medium-Dose Interferon Alpha 2B in Stages IIB, IIC, and III Cutaneous Melanoma Patients: A Single Institution, Randomized Study. Front Immunol 8:625).

M-Vax™ is an active immunotherapy based on the modification of autologous cancer cells with the hapten dinitrophenyl (DNP). The treatment program consists of multiple intradermal injections of DNP-modified autologous tumor cells mixed with BCG. Conducted trials showed partial and mixed clinical responses. To prepare vaccines, tumor cells are irradiated and then modified with DNP by a standard method. All M-Vax vaccines contained live tumor cells, dead tumor cells, and lymphocytes (Berd. 2004. M-Vax: an autologous, hapten-modified vaccine for human cancer. Expert Rev Vaccines. 3:521-527).

Another whole tumor cell vaccine used for treating breast cancer consists in the cell lines T47D (HER2^{low}) and SKBR3 (HER2^{high}) genetically modified by plasmid DNA transfection to secrete GM-CSF. The vaccine cells are resuspended in serum-free medium, cryopreserved, irradiated, thawed and mixed to create an HER2-positive vaccine that secreted GM-CSF levels of 305 ng/10⁶ cells/24 hours (Emens et al. 2009. Timed sequential treatment with cyclophosphamide, doxorubicin, and an allogeneic granulocyte-macrophage colony-stimulating factor-secreting breast tumor vaccine: a chemotherapy dose-ranging factorial study of safety and immune activation. J Clin Oncol 27:5911-5918). GVAX™ is another vaccine composed of whole tumor cells (allogeneic or autologous) genetically modified to secrete GM-CSF and then irradiated (Hege et al. 2006. GM-CSF gene-modified cancer cell immunotherapies: of mice and men. Int Rev Immunol 25:321-352).

Other group developed an allogeneic whole tumor cell vaccine derived from a HLA-A*0201 renal cancer tumor cell line (RCC26) transfected with the human genes from IL-7 and CD80 (B7.1). This genetically-modified vaccine (RCC26/IL7/CD80) is frozen in the presence of HBSS, 7.5% dimethyl sulfoxide and 20% human serum albumin. The cryopreserved aliquots of the vaccine are then irradiated (120 Gy) according to a standardized protocol which completely prevented long-term survival of tumor cells in vitro (Westermann et al. 2011. Allogeneic gene-modified tumor cells (RCC-26/IL-7/CD80) as a vaccine in patients with metastatic renal cell cancer: a clinical phase-I study. Gene Ther 18:354-63).

In addition, an autologous tumor lysate vaccine was manufactured from surgically resected tumors and administered subcutaneously together with GM-CSF. The fresh tumor specimens were lysed by physical mincing followed by alternating freeze-thawing for five cycles by freezing the minced material to −80° C. and then briefly thawing in a water bath five times. During the vaccination period the vaccine was administered with GM-CSF as a single subcutaneous (s.c.) injection over the deltoid on day 1 followed by a further s.c. administration of GM-CSF into the vaccine site (Powell et al. 2006. Recombinant GM-CSF plus autologous tumor cells as a vaccine for patients with mesothelioma. Lung Cancer 52:189-197).

Chiang et al. compared methods for preparing autologous and allogeneic tumor lysates by UVB irradiation (UVB-L) and freeze-thaw cycles (FTL) and conducted a pilot study in recurrent ovarian cancer subjects using HOCI-oxidized autologous whole tumor lysate-pulsed DC to induce rapid necrosis and increase the immunogenicity of tumor cells. DCs engulfed HOCI-oxidized lysate most efficiently, stimulated robust mixed leukocyte reactions (MLRs) and elicited strong tumor-specific IFN-g secretions in autologous T cells. (Chian et al. 2013. A dendritic cell vaccine pulsed with autologous hypochlorous acid-oxidized ovarian cancer lysate primes effective broad antitumor immunity: from bench to bedside. Clin Cancer Res 19:4801-4815).

Another vaccine named Melacine includes a mixture of mechanical lysates from two allogeneic melanoma cell lines co-administered with an immunologic adjuvant (DETOX). Both melanoma cell lines that comprise the cell lysate were generated from two different patients. These lysates were generated by mechanical disruption and three cycles of freeze—thawing. The adjuvant DETOX was comprised of a mixture of monophosphoryl lipid A (detoxified endotoxin) from Salmonella Minnesota, cell wall skeleton from Mycobacterium phlei, squalene and an emulsifier. (Sondak et al. 2003. Results of clinical trials with an allogeneic melanoma tumor cell lysate vaccine: Melacine. Semin Cancer Biol 13:409-415).

Patients with stage IV solid malignancies were treated in cohorts that received 10⁶, 10⁷, and 10⁸ DCs intradermally (i.d.) every 2 weeks for three vaccines. Each vaccine was composed of a mixture of half DCs pulsed with autologous tumor lysate and the other half with KLH. Peripheral blood mononuclear cells (PBMCs) harvested 1 month after the last immunization was compared with pretreatment PBMCs for immunological response. Delayed-type hypersensitivity reactivity to tumor antigen and KLH was also assessed. (Chang et al. 2002. A phase I trial of tumor lysate-pulsed dendritic cells in the treatment of advanced cancer. Clin Cancer Res 8:1021-1032).

However, the formulation of all these whole-tumor cell vaccines does not include the pre-treatment of tumor cells (neither cell lines or autologous tumors) with non-lethal heat-shock and the subsequent production of the DAMP-rich cell lysates. Moreover, none of them used hemocyanins as adjuvants, with the exception of the ex vivo tumor-loaded DC vaccines. These and other deficiencies in the previous therapies are overcome by the provision of immunogenic formulations (LCVX) based of cell lysates from heat-shock conditioned tumor cell populations, combined with a immunologically effective amount of adjuvant, of the present invention.

### SUMMARY OF THE INVENTION

The present invention provides an immunogenic formulation (LCVX) for treating cancer in a subject, comprising:
i) An immunologically effective amount of two or more cell lysates generated from Heat Shock-Conditioned Cancer Cell populations, wherein each heat shock-conditioned cancer cell population immediately before lysis (a) expressed two or more tumor-associated antigens (TAAs), (b) had elevated levels of two or more Damage-Associated Molecular Patterns (DAMPs) and (c) had a cell viability of higher than 80%; and
ii) An immunologically effective amount of an adjuvant.

Thus, the invention provides an immunogenic formulation capable to improve DC capacity to cross-present TAAs for treating cancer in a subject.

Moreover, the invention provides an immunogenic formulation capable to improve CD3⁺ and CD8⁺ T-cell infiltration of tumors inhibiting tumor growth in a mammalian model.

In one embodiment, the cell viability may be assessed by the absence of necrotic or apoptotic signals.

Alternatively or in addition, the combined two or more cell lysates may comprise at least three TAAs and at least three DAMPs at elevated levels.

In one embodiment, the adjuvant may be selected from the group consisting of glycosylated adjuvant, a carrier adjuvant, a Very Small Size Proteoliposome adjuvant (VSSP), an oil-in-water emulsion, a saponin-based adjuvant, a mineral salt adjuvant, an immunostimulant, and any combinations thereof. In one example, where the adjuvant comprises a glycosylated adjuvant, the glycosylated adjuvant may be a particular hemocyanin or combinations of particular hemocyanins. As an example, the particular hemocyanin may be obtained from mollusk, preferably species from Muricidae, Fissurellidae and Haliotidae families. Specifically, the particular hemocyanin may be Keyhole limpet hemocyanin (KLH), Concholepas concholepas hemocyanin (CCH), or Fissureulla latimarginata hemocyanin (FLH).

In one embodiment, the immunogenic formulation may comprise at least 0.5 micrograms of the particular hemocyanin per dose. For example, the immunogenic formulation may comprise from 0.5 micrograms to 500 micrograms, optionally from 5 micrograms to 150 micrograms, or optionally about 150 micrograms, of the particular hemocyanin per dose.

In another embodiment, the adjuvant may comprise a carrier adjuvant, optionally a liposome or a virosome. For example, where the adjuvant comprises a liposome, the immunogenic formulation may comprise from 0.5 microgram to 200 microgram of the liposome per dose.

Alternatively, the adjuvant may comprise a virosome and the immunogenic formulation may comprise from 0.1 micrograms to 5 mg of viral protein of the virosome per dose.

In another embodiment, the adjuvant may comprise a VSSP, optionally a ganglioside M3 (GM3), and optionally the immunogenic formulation comprises from 10 micrograms to 300 micrograms of GM3 per dose.

In another embodiment, the adjuvant may comprise an oil-in-water adjuvant, optionally MF59 or montanide.

Optionally, the adjuvant may comprise MF59 and the immunogenic formulation may comprise from 0.2% to 20% (vol/vol) of MF59.

Optionally, the adjuvant may comprise montanide and the immunogenic formulation may comprise from 2% to 70% (vol/vol) of montanide.

In another embodiment, the adjuvant may comprise a saponin-based adjuvant, optionally immunostimulatory complexes (ISCOMs) or Quillaja saponaria-21 (QS-21).

Optionally, the adjuvant may comprise ISCOMs and the immunogenic formulation may comprise from 0.5 micrograms to 50 micrograms of ISCOMs per dose.

Optionally, the adjuvant may comprise QS-21 and the immunogenic formulation may comprise from 0.01 micrograms to 30 micrograms of QS-21 per dose.

In another embodiment, the adjuvant may comprise a mineral salt adjuvant, optionally alum, aluminum salt and TLR4 agonist-based adjuvant, optionally AS01, AS02, AS03, AS04, or AS15.

Optionally, the adjuvant may comprise alum or an aluminum salt and the immunogenic formulation may comprise from 1 microgram to 50 mg of alum or the aluminum salt per dose.

Optionally, the adjuvant may comprise TLR4 agonist-based adjuvant, optionally AS01, AS02, AS03, AS04, or AS15, and the immunogenic formulation may comprise from 0.1 micrograms to 20 micrograms of TLR4 agonist-based adjuvant, optionally AS01, AS02, AS03, AS04, or AS15, per dose.

In another embodiment, the adjuvant may comprise an immunostimulant, optionally a Toll-like receptor (TLR) ligands (optionally, Poly I:C, poly-ICLC, monophosphoryl lipid A (MPL), glucopyranosyl lipid adjuvant (GLA), imiquimod, or CpG ODN) or polysaccharides (optionally, chitin, chitosan, or β-glucan).

Optionally, the adjuvant may comprise Poly I:C or poly-ICLC and the immunogenic formulation may comprise from 0.1 mg to 10 mg of Poly I:C or poly-ICLC per dose.

Optionally, the adjuvant may comprise MPL and the immunogenic formulation may comprise from 5 micrograms to 500 micrograms of MPL per dose.

Optionally, the adjuvant may comprise GLA and the immunogenic formulation may comprise from 0.5 micrograms to 50 micrograms of GLA per dose.

Optionally, the adjuvant may comprise imiquimod and the immunogenic formulation may comprise from 25 mg to 500 mg of imiquimod per dose.

Optionally, the adjuvant may comprise CpG ODN and the immunogenic formulation may comprise from 50 micrograms to 10 mg of CpG ODN per dose.

Optionally, the adjuvant may comprise chitin or chitosan and the immunogenic formulation may comprise from 0.01 mg to 100 mg of chitin or chitosan per dose.

Optionally, the adjuvant may comprise β-glucan and the immunogenic formulation may comprise from 0.1 mg to 500 mg of β-glucan per dose.

In one embodiment, the two or more DAMPs may be selected from the group consisting of post-heat shock and pre-lysis secretion of: chromatin-associated protein high-mobility group box 1 protein (HMGB1), ATP, S100/Calgranulin protein family members [optionally, S100 calcium binding protein A8 (S100A8), S100 calcium binding protein A9 (S100A9), and/or S100A12/EN-RAGE], Heat shock protein (HSP) 70 (HSP70), HSP90, HSP60, HSP72, nucleic acids (optionally mitochondrial DNA, dsDNA, and dsRNA), Prostaglandin E2 (PGE2), Monosodium urate (MSU), uric acid, and Peroxiredoxin 1 (Prx1), pre-lysis plasma membrane expression of: Calreticulin (CRT) and Death domain 1 alpha (DD1alpha); and elevated levels of HSP70, HSP72, HSP60, and HSP72.

Alternatively or in addition, the at least three DAMPs include one, two, or all three of post-heat shock and pre-lysis secretion of HMGB1 and/or lysis plasma membrane expression of CRT.

Alternatively or in addition, the at least three DAMPs are in an effective amount to induce activation and maturation of antigen-presenting cells (APCs) when administered to a subject.

In one embodiment, the two or more TAAs may be selected from the group consisting of melanoma antigen recognized by T cells 1 (MART1), glycoprotein 100 (gp100), tyrosinase, New York esophageal squamous cell carcinoma 1 (NY-ESO-1), melanoma-associated antigen 1 (MAGE1), melanoma-associated antigen 2 (MAGE2), melanoma-associated antigen 3 (MAGE3), melanocortin 1 receptor (MC1R), melanoma-associated chondroitin sulfate proteoglycan (MCSP), survivin, human epidermal growth factor receptor (Her2), carbohydrate antigen (CA) 19-9 (CA10-9), mucin 1 (MUC1), mucin 5AC (MUC5AC), carcinoembryonic antigen (CEA), G antigen 1 (GAGE1), G antigen 2 (GAGE2), B melanoma antigen (BAGE), cytokeratin 7 (CK7), Cytokeratin 19 (CK19), and cancer antigen 125 (CA125).

In one embodiment, the two or more cell lysates may be in an amount effective to induce the release of two or more proinflammatory cytokines selected from the list consisting of IL-6, IL-8, TNF-α, IL-10, IL-1, IFN-γ, and IL-12.

In one embodiment, the two or more cell lysates may be in an amount effective to induce the release of TNF-α and IL-12.

In one embodiment, the two or more cell lysates are in an amount effective to induce the overexpression of three or more maturation-associated markers on antigen-presenting cell membrane selected from the group consisting of MHC class I, MHC class II, CD83, CD86, CD80, CD40, CCR7, DEC-205, DC-SIGN and MICA.

In one embodiment, the two or more cell lysates may be in an amount effective to induce the overexpression of CD83, CD86, and CD80.

In one embodiment, the two or more cell lysates may be in an amount effective to improve dendritic cells (DCs) capacity to cross-present TAAs.

In one embodiment, the immunogenic formulation may comprise each cell lysate produced from at least 50,000 cells per dose.

In one embodiment, the immunogenic formulation may comprise a total cell lysate produced from 100,000 to 50,000,000 cells per dose, optionally about 5,000,000 cells per dose.

In one embodiment, at least one of the two or more cell lysates may be generated from cancer cell lines selected from the group consisting of malignant melanoma, prostate cancer, gallbladder cancer, lung cancer, breast cancer, colon cancer, kidney cancer, kidney cancer, cervical cancer, ovarian cancer, gastric cancer, brain cancer, and pancreatic cancer.

In one embodiment, at least one of the two or more cell lysates may be generated from fresh metastatic tumor tissues.

Optionally, the fresh metastatic tumor tissue may be obtained from the subject to be treated.

In one embodiment, the two or more cell lysates may be autologous, allogeneic, or combinations with respect to the subject to be treated.

In one embodiment, the immunogenic formulation may be suitable for administration by subcutaneous, intradermal, intratumoral, or intranodal injection.

In yet another object, the invention provides a method of generating the immunogenic formulation as described herein comprising admixing the two or more cell lysates and the immunologically effective amount of the adjuvant.

In one embodiment, the two or more cell lysates may be admixed before addition of the immunologically effective amount of the adjuvant.

Optionally, the two or more cell lysates may be admixed with the immunologically effective amount of the adjuvant immediately prior to administration to the subject or up to 48 hours after admixture.

In one embodiment, the two or more cell lysates and optionally the adjuvant may be lyophilized and stored prior to use and may be reconstituted by admixture with the immunologically effective amount of the adjuvant immediately or with sterile water immediately prior to administration to the subject or up to 48 hours after admixture.

In one embodiment, at least one of the two or more cell lysates may be generated by:
i) providing a cancer cell line that expresses the two or more TAAs;
ii) incubating the cancer cell line at a temperature and for a time sufficient to induce heat shock to produce a population of heat-shocked cancer cells;
iii) incubating the population of heat-shocked cancer cells at 37° C. for a time sufficient to induce the elevated levels of the two or more DAMPS to produce the heat shock-conditioned cancer cell population with the cell viability of higher than 80%;
iv) disrupting the heat shock-conditioned cancer cell population to produce at least one cell lysate, optionally by mechanical disruption, sonication, microwave, blenders, standard liquid homogenizers, or high pressure homogenizers; and
v) homogenizing the at least one cell lysate.

Optionally, the cancer cell line may be provided by obtaining cells from fresh metastatic tumor tissue. For example, the fresh metastatic tumor tissue may be obtained from the subject.

Optionally, the cancer cell line may be provided by screening one or more cancer cell lines for expression of the two or more TAAs and selecting the cancer cell line that expresses the two or more TAAs.

Optionally, the temperature sufficient to induce heat shock may be between 39° C. and 45° C., optionally at about 42° C.

Optionally, the time sufficient to induce heat shock may be between 15 minutes and 3 hours, optionally about 1 hour.

Optionally, the cells in step ii) may be in a serum-free, red-phenol-free culture medium, optionally AIM-V red phenol-free or PBS+human serum albumin (0.1-5%).

Optionally, the time to induce the elevated levels of the two or more DAMPs is from 0.5 hours to 6 hours, optionally from 1 to 3 hours, and optionally about 2 hours.

Optionally, after step iii), the heat shock-conditioned cancer cell population may be screened for expression of the two or more DAMPs and steps i)-iii) may be repeated if the heat shock-conditioned cancer cell population does not express the two or more DAMPs.

Optionally, after step iv), the at least one cell lysate may be screened for presence of the two or more DAMPs and steps i)-iv) may be repeated if the at least one cell lysate does not comprise the two or more DAMPs.

Optionally, the heat shock-conditioned cancer cell population may be admixed with one or more additional heat shock-conditioned cancer cell populations that (a) express two or more TAAs, (b) have two or more DAMPs, and (c) have a cell viability of higher than 80% before step iv).

Optionally, the disrupting may comprise at least one cycle of freezing and thawing of the heat shock-conditioned cancer cell population.

Optionally, the disrupting may comprise 2 to 4 cycles of cycle of freezing and thawing of the heat shock-conditioned cancer cell population. For example, the freezing may be with liquid nitrogen.

Optionally, the thawing may be at 35-40° C., optionally 37° C.

In one embodiment, the method may further comprise a sterilizing step after the incubating step iii).

In another embodiment, the sterilizing step may be after the homogenizing step v).

Optionally, the sterilizing step may comprise irradiation.

Optionally, the irradiation may comprise a dose from 50 to 100 Gy, optionally 80 Gy.

In one embodiment, the method may further comprise testing the at least one cell lysate for inducing the activation of APCs to display a phenotype similar to mature DCs, optionally as tested by cell surface marker expression and cytokine release.

The present invention also encompasses a method of treatment of a cancer in a subject comprising administering the immunogenic formulation of any one of claims 1-47 to the subject. Expressed in another way, the present invention resides in the immunogenic formulation of any one of claims 1-47 for use in the treatment of a cancer in a subject. The invention may also be expressed as use of the immunogenic formulation of any one of claims 1-47 for use in the manufacture or a medicament for the treatment of a cancer in a subject.

In one embodiment, the cancer may be selected from the group consisting of melanoma, malignant melanoma, prostate cancer, gallbladder cancer, lung cancer, breast cancer, colon cancer, kidney cancer, renal cancer, cervix cancer, ovarian cancer, gastric cancer, brain cancer, and pancreatic cancer.

In one embodiment, the immunologically effective amount of two or more cell lysates and immunologically effective amount of an adjuvant of the immunogenic formulation may be administered separately to the subject at the same time or at different times.

In one embodiment, the method of treatment may further comprise administering an immune check-point inhibitor agent before, after, or simultaneously with the administration of the immunogenic formulation.

Optionally, the immune check-point inhibitor agent may inhibit PD-1, PD-L1 or CTLA4.

Optionally, the immune check-point inhibitor agent is a monoclonal antibody. In one example, the monoclonal antibody may be selected from the group consisting of: pembrolizumab, nivolumab, cemiplimab, atezolizumab, avelumab, durvalumab, ipilimumab, or a biosimilar thereof.

Other objects of the present invention will be apparent from the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the selection of tumor cell lines based on the expression of tumor associated antigens. Summary of tumor associated antigen (TAA) expression in gallbladder cancer (GBC) cell lines. The arrows indicate the cell lines chosen to manufacture immunogenic tumor lysate. ND: not determined.

FIG. 2 shows the selection of tumor cells based on the level of heat-shock inducible damage associated molecular patterns (DAMPs). The levels of ATP (a) or HMGB1 (b) were evaluated in the supernatants from heat shock-treated or control melanoma (Mel1, Mel2, Mel3) or gallbladder cancer (GBC) cell lines and tissues. Bars represent the averages and standard deviations of at least three independent experiments. * p<0.05; ** p<0.01; *** p<0.001; **** p<0.0001. (c) Representative histograms showing the extracellular expression levels of translocated calreticulin (eCRT) in heat shock-treated (dark grey) or control (light grey) melanoma and GBC cells. White histograms indicate isotype control staining. The percentage of eCRT positive (eCRTpos) for each condition is shown.

FIG. 3 shows that the heat shock treatment of both a mix of three melanoma cell lines or eight different gallbladder cancer cell lines (GBCCLs), does not significantly impair cell viability. An equitative mix of three melanoma cell lines (Mel1+Mel2+Mel3) (A, B) or each melanoma cell line individually (B) were subjected to our heat shock treatment: 1 hour to 42° C. +2 hours to 37° C. [HS (42° C.)], a more aggressive HS treatment: 2 hours to 46° C. [HS (46° C.)], to three cycles of freeze and thaw (F/T), or to a control (Ctrl) condition (37° C. for 3 hours). A) Representative dot plots showing the percentage of live cells (LIVE/DEAD® exclusion) for each condition for Mel1+Mel2+Mel3 mix. B) Bars represent the average and SD of live cells for Me11+Me12+Me13 mix or each cell treated individually. Data are representative of two independent experiments. C) Eight GBCCLs (TGBC-1TKB, −2TKB, −14TKB, −24TKB, NOZ, GBd1, G415, and OCUG1) were subjected to our heat shock treatment: 1 hour to 42° C. +2 hours to 37° C. [HS (42° C.)], or to a control (Ctrl) condition (37° C. for 3 hours). Bars represent the average and SD of live cells (LIVE/DEAD exclusion) relative to Ctrl conditions (100%). Data are representative of six independent experiments.

FIG. 4 shows the selection of heat shock-conditioned tumor cell lysate mixtures, based on the induction of differentiation of activated monocytes into mature DCs. Surface expression of HLA-DR, CD80, CD86 (a, c), and HLA-ABC, CD83, and CCR7 (b) was evaluated by flow cytometry in activated monocytes (AM) incubated or not for 24 hours with 100 μg/mL of heat shock-conditioned tumor lysates generated from individual gallbladder cancer cell lines (GBCCLs) (c) or different mixtures (M1-M8) of three different GBCCLs (a, b). Bars represent the average and SD of the fold induction of mean fluorescence intensity (MFI) for each marker relative to AM from at least three independent experiments. * p<0.05; ** p<0.01; *** p<0.001; **** p<0.0001.

FIG. 5 shows the T cell activation by autologous monocyte-derived DCs loaded with a heat shock conditioned GBC lysate recognize HLA-A2-matched GBC cell lines. (a-c) Purified CD3+ T cells were co-cultured for 14 days with autologous HLA-A2+ AM, TRIMEL-DCs, M2-DCs or cultured alone. The surface expression of CD25, CD69, CXCR3 and CXCR4 (a, b) were evaluated in the CD4+ (a) and CD8+ (b) T cells populations by flow cytometry. Bars represent the average and SD from at least three independent experiments of the % of T cells positive for each marker, with the exception of CXCR3 and CXCR4 data that are shown as fold induction of the mean fluorescence intenstity (MFI) relative to unstimulated T cells. * p<0.05; ** p<0.01; *** p<0.001 (comparison vs unstimulated T cells). (c) Sorted CD8+ T cells were challenged for 16 hours with the HLA-A2+ GBCCL 2TKB, GBd1, CAVE, the melanoma cell line Mer1 or K562 cells. IFN-γ release was measured by ELISPOT at different effector:target ratios as indicated. Data represent the average and SD of at least three independent experiments. * p<0.05; *** p<0.001; **** p<0.0001 (comparison M2-DC versus TRIMEL-DCs stimulated T cells).

FIG. 6 shows that the heat shock conditioned human melanoma cell lysate (TRIMEL) induced murine DC maturation in vitro. DCs isolated by positive selection from spleens of C56BL/6 mice were incubated for 24 hours with: LPS, TRIMEL, heat shock conditioned B16F10 cell lysate (HS-lysate), a 1:1 mixture of TRIMEL+B16F10 HS-lysate, or keeped non-activated (NA). Representative histograms for the MFI for MHC-II and CD86 are showed for conventional DCs (cDCs) (A) or plasmocytoid DCs (pDCs) (C). Quantification of three independent experiments for the fold induction relative to NA are shown for each marker for cDCs (B) or pDCs (D). E) Intracellular levels of IL-12 were determined in total spleen DCs. The fold induction relative to NA is shown. * p<0.05; ** p<0.01.

FIG. 7 shows the inhibition of B16F10 tumor growth by prophylactic treatment with Lycellvax. A) Schematic representation of the protocol of prophylactic treatment with LCVXLCVX. C57BL/6 female mice were inoculated s.c. at days −19, −9 and −2 (before tumor challenging) with: i) LCVX (Mel) (TRIMEL+B16F10 HS lysate+CCH), ii) Lysates (TRIMEL+B16F10 HS lysate), iii) CCH, or iv) vehicle (PBS). At the day 0, mice were challenged (s.c.) with 1.5×10⁵ B16F10 cells and tumor growth was monitored every 2 days for 18 days post-tumor challenging. B) Tumor growth curves of individual mice are shown for the four different treatment groups. C) Average tumor sizes and SD of the mean per group from experiment shown in panel (A). Statistical analysis was performed with two-way ANOVA after Bonferroni correction. ** p<0.01.

FIG. 8 shows the inhibition of B16F10 tumor growth by therapeutic treatment with LCVX. A) Schematic representation of the protocol of therapeutic treatment with LCVX. C57BL/6 female mice were challenged s.c. at day 0 with 0.25×10⁵ B16F10 cells and then inoculated s.c. at days 1, 6 and 12 post-tumor challenging with: i) LCVX (Mel) (TRIMEL+B16F10 HS lysate+CCH), ii) Lysates (TRIMEL+B16F10 HS lysate), iii) CCH, or iv) vehicle (PBS). Tumor growth was monitored every 2 days for 19 days post-tumor challenging. B) Tumor growth curves of individual mice are shown for the four different treatment groups. C) Average tumor sizes and SD of the mean per group from experiment shown in panel (A). Statistical analysis was performed with two-way ANOVA after Bonferroni correction. * p<0.05.

FIG. 9 shows that the LCVX-mediated tumor growth inhibition depends on adaptive immune cells. C57BL/6 or immunodeficient NODSCID female mice were inoculated s.c. at days −19, −9 and −2 (before tumor challenging) with: i) LCVX (Mel) (TRIMEL+B16F10 HS lysate+CCH), or ii) vehicle (PBS). At the day 0, mice were challenged (s.c.) with 1.5×10⁵ B16F10 cells and tumor growth was monitored every 2 days for 17 days post-tumor challenging. Average tumor sizes and SD of the mean of each experimental group are shown. ns: no significant difference in tumor growth between NODSCID mice treated with LCVX or PBS.

FIG. 10 shows the evaluation of lysate dilution, B16F10-derived antigens and different hemocyanin adjuvants in the tumor protective activity of LCVX. C57BL/6 female mice were inoculated s.c. at days −19, −9 and −2 (before tumor challenging) with: A) two different doses of LCVX: 1 mg of lysate protein/dose/animal (LCVX (Mel)), or 0.1 mg of lysate protein/dose/animal (0.1 LCVX (Mel)), or with vehicle (PBS); B) TRIMEL (Lysate 2), TRIMEL+CCH (Lysate 2 CCH), or PBS; C) (TRIMEL+B16F10 HS lysate+CCH), LCVX (Mel)-CCH, (TRIMEL+B16F10 HS lysate+FLH), LCVX (Mel)-FLH, CCH or FLH alone, or PBS. At the day 0, mice were challenged (s.c.) with 1.5×10⁵ B16F10 cells and tumor growth was monitored every 2 days for 18-20 days post-tumor challenging. Average tumor sizes and SD of the mean of each experimental group are shown. Statistical analysis was performed with two-way ANOVA after Bonferroni correction. * p<0.05. ns: no significant difference.

FIG. 11 A) shows that a heat shock-conditioned human gallbladder cancer cells (GBC) lysate vaccine inhibits B16F10 tumor growth. C57BL/6 female mice were inoculated s.c. at days −19, −9 and −2 (before tumor challenging) with: i) LCVX (MEL) (TRIMEL+B16F10 HS lysate+CCH), ii) LCVX (GBC) (M2 GBC cell lysate+B16F10 HS lysate+CCH), or iii) vehicle (PBS). At the day 0, mice were challenged (s.c.) with 1.5×10⁵ B16F10 cells and tumor growth was monitored every 2 days for 19 days post-tumor challenging. Tumor growth curves of individual mice are shown for the three different treatment groups. Average tumor sizes and SD of the mean of each experimental group at day 19 are shown. B) Shows that LCVX can inhibit the tumor growth of a murine colon adenocarcinoma MC38. C57BL/6 female mice were challenged s.c. at day 0 with 0.25×10⁵ MC38 cells and then inoculated s.c. at days 1, 6 and 12 post-tumor challenging with: i) LCVX (Col) (TRIMEL+MC38 HS lysate+CCH), ii) Lysates (TRIMEL+MC38 HS lysate), iii) CCH alone or iv) vehicle (PBS). Tumor growth was monitored every 2 days for 26 days post-tumor challenging. Tumor growth curves of individual mice are shown for the three different treatment groups. Average tumor sizes and SD of the mean of each experimental group at day 26 are shown. Statistical analysis was performed with two-way ANOVA after Bonferroni correction. * p<0.05.

FIG. 12 shows that the anti-tumor effect of anti-PD-1 is improved by combination with LCVX therapy. A) Schematic representation of the protocol of therapeutic treatment with combination of anti-PD-1 and LCVX. C57BL/6 female mice were challenged s.c. at day 0 with 0.25×10⁵ B16F10 cells and then inoculated s.c. at days 1, 6 and 12 post-tumor challenging with: LCVX (TRIMEL+B16F10 HS lysate+CCH), or vehicle (PBS). Additionally, mice received three i.p. doses of anti-PD-1 antibodies (or vehicle PBS) at days 4, 7 and 11 post-tumor challenging. Tumor growth and survival of mice were monitored every 2 days for 18 or 35 days post-tumor challenging, respectively. B) Tumor growth curves of individual mice are shown for the four different treatment groups shown in panel (A). C) Average tumor sizes and SD of the mean per group from experiment shown in panel (A). D) Kaplan-Meier curves showing the percent of survival of mice of the four different treatment groups shown in panel (A). Statistical analysis was performed with two-way ANOVA after Bonferroni correction. ** p<0.01; *** p<0.001.

FIG. 13 shows that LCVX therapy increases CD8⁺ T cell infiltration into melanoma tumors and enhances CD3⁺, CD4⁺ and CD8⁺ T cell infiltration in anti PD1 treated mice. A) Example of immunohistochemical analysis of B16F10 tumors obtained from mice treated with PBS, anti PD1, LCVX, or anti PD1+ LCVX. C57BL/6 female mice were challenged s.c. at day 0 with 0.25×10⁵ B16F10 cells and then inoculated s.c. at days 1, 6 and 12 post-tumor challenging with: LCVX (TRIMEL+B16F10 HS lysate+CCH), or vehicle (PBS). Additionally, mice received three i.p. doses of anti-PD-1 antibodies (or vehicle PBS) at days 4, 7 and 11 post-tumor challenging. Mice were sacrificed day 15 and tumors fixed and analyzed by IHC. B) IHC Quantification of CD3⁺, CD4⁺ and CD8⁺ T cell infiltration of melanoma tumors. Twenty different fields of samples obtained from 3 mice per group of treatment were analyzed at 60× and number of positive T cells counted. Statistical analysis was performed with two-way ANOVA after Bonferroni correction. *** p<0.001; **** p<0.0001.

### DETAILED DESCRIPTION

The terms “patient” or “subject” refer to mammals including humans, primates, rabbits, rats, mice, and other animals.

The terms “treating” and “treatment” refer to an approach for obtaining beneficial or desired clinical results. For the purpose of this invention, the approach comprises the administration of lysates from conditioned tumor cell lines of the present invention to prevent or delay the onset of the symptoms, complications, or biochemical indicia of a disease, alleviating the symptoms or preventing or delaying spread (e.g., metastasis, for example metastasis to the lung or to the lymph node) or arresting or inhibiting further development of cancer in a subject. The treatment may be prophylactic (to prevent or delay the onset of the disease, or to prevent the manifestation of clinical or subclinical symptoms thereof) or therapeutic suppression or alleviation of symptoms after the manifestation of the disease.

The term “conditioned” refers to heat-shocked tumor cell lines by a first incubation at temperature between 39 and 45° C. for a short time period, followed by a second incubation at 37° C., and maintaining a cell viability higher than 80% in absence of necrotic or apoptotic signals.

The term “Tumor Associated Antigens” or TAAs refers, but not limited to proteins that can be recognized by the immune system such as: MART1, gp100, tyrosinase, NY-ESO-1, MAGE1, MAGE2, MAGE3, MC1R, MCSP, survivin, Her2/Neu, CA19-9, MUC1, CEA, GAGE1/2, BAGE.

The term “Damage Associated Molecular Patterns” or DAMPs refers, but not limited to HMGB1, ATP, CRT and/or other heat shock proteins capable to induce the activation and maturation of APCs.

The term DC release of proinflammatory cytokines IL-6, IL-8, TNF and/or IL-12, and the overexpression of maturation-associated markers on antigen-presenting cell membrane including MHC class I, MHC class II, CD83, CD86, CD80 and/or CD40.

The phrase “immune cell response’ refers to the response of immune system cells to external or internal stimuli, including but not limited to antigen, cytokines, chemokines, and other cells producing biochemical changes in the immune cells that result in immune cell migration, killing of target cells, phagocytosis, production of antibodies, other soluble effectors of the immune response, and the like.

The term “cancer” refers, but not limited to malign melanoma, gallbladder cancer, prostate cancer, lung cancer, breast cancer, colon cancer, kidney cancer, cervix cancer, gastric cancer.

The term “conditioned tumor cell lines” refers, but not limited to tumor cell lines from melanoma, gallbladder cancer, prostate cancer, lung cancer, breast cancer, colon cancer, kidney cancer, cervix cancer, gastric cancer, ovary cancer, pancreatic cancer, conditioned to heat shock by a first incubation at temperature between 39 and 45° C. for a short time period, followed by a second incubation at 37° C., and maintaining a cell viability higher than 80% in absence of necrotic or apoptotic signals and the presence of detectable levels of immunostimulatory danger signals.

The term “lysate” refers to the cell derived product resulting from the lysis of cells by three repeated cycles of freeze/thaw using liquid nitrogen.

The term “glycosylated adjuvant” refers to, but not limited to mollusk hemocyanin from species of Muricidae, Fissurellidae and Haliotidae families.

The term “antigen presenting cell (APC)” refers to monocyte derived cells stimulated with CTLC with expression of molecular markers of mature DCs and capability to induce activation of T cells by presentation of tumor derived antigens.

In one aspect, it is an object of the present invention to provide an immunogenic formulation comprising an immune stimulant and glycosylated adjuvant including but not limited to mollusk hemocyanin from species of Muricidae, Fissurellidae and Haliotidae families.

An integral and essential part of this invention is the use of an immunogenic formulation for treating cancer in a subject, comprising:
i) an immunologically effective amount of two or more cell lysates generated from heat shock-conditioned cancer cell populations, wherein each heat shock-conditioned cancer cell population immediately before lysis (a) expressed two or more tumor-associated antigens (TAAs), (b) had elevated levels of two or more damage-associated molecular pattern molecules (DAMPs), and (c) had a cell viability of higher than 80%; and
ii) an immunologically effective amount of an adjuvant.

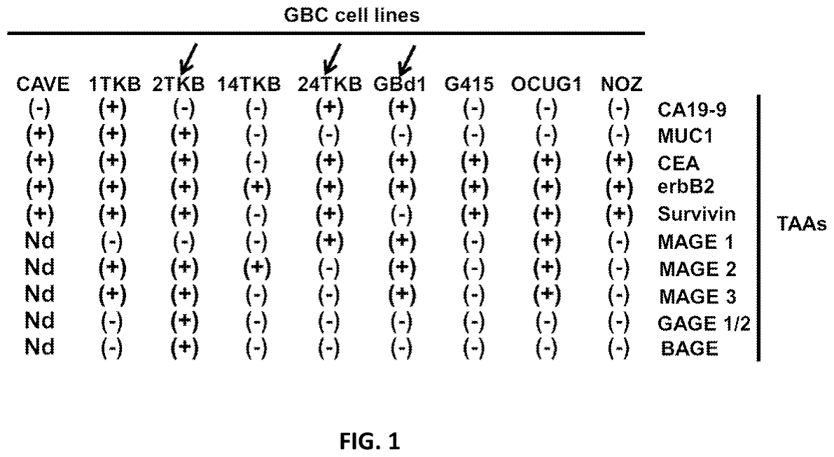

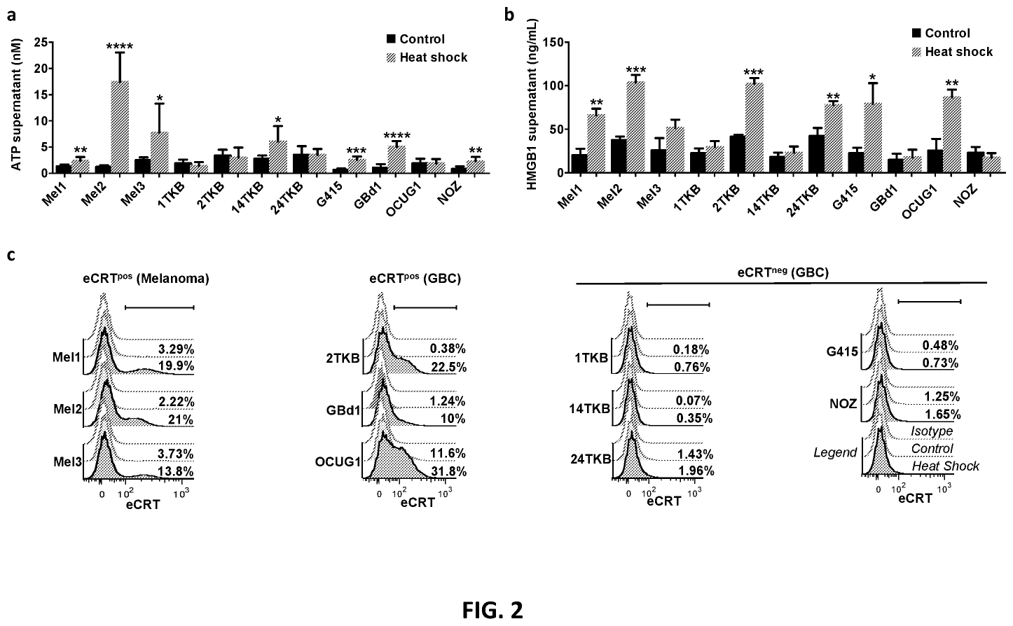

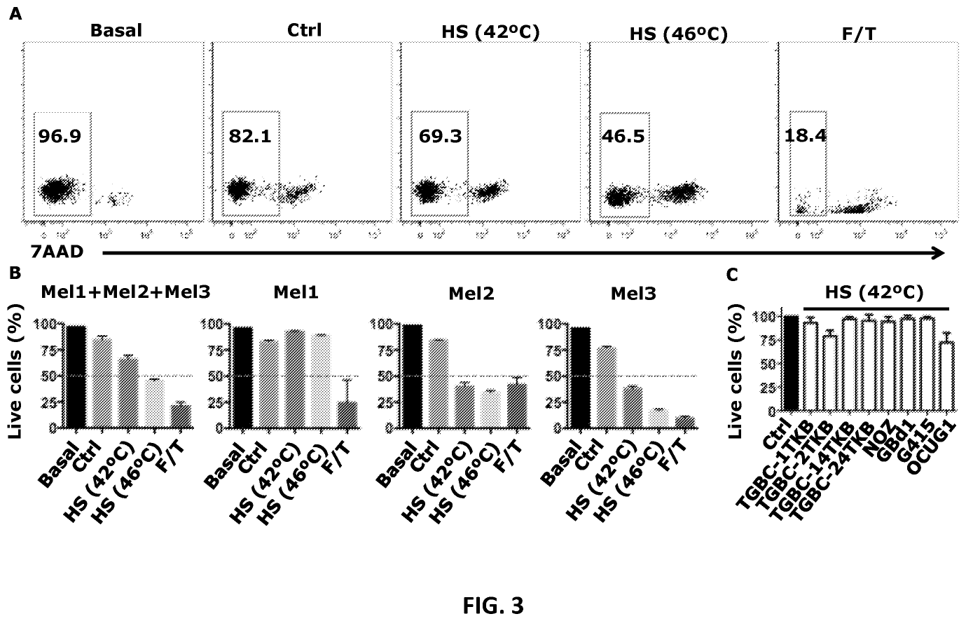

In an alternative provided by the invention, the lysate of tumor cells is obtained from fresh tumor cell derived from patients with different kinds of cancer combined or not with lysate of allogeneic heat shock conditioned tumor cell lines of the same tumor type. The phenotype of used cells may be confirmed through conventional techniques in order to determine the expression of TAAs (FIG. 1). The cells are resuspended to a density between 4×10⁶-15×10⁶ cells/mL, preferentially near to 10×10⁶ cells/mL, or tissues are then incubated between 15 minutes and 4 hours, with a preferred timing of 1 and 3 hours ideally around 2 hours at a temperature that range between 39 and 44° C., more preferably between 40 and 43° C. and preferentially near 42° C., in a serum-free culture medium. Later, the cells and/or tissues are placed at physiological temperature again, that is, around 37° C. for 1 to 6 hours, ideally between 2 and 4 hours preferentially 3 hours before being lysate. This heat shock incubation of tumor cell lines should induce the accumulation of different DAMPs, such as HMGB1 and ATP release and CRT translocation (FIG. 2). Of note, it is preferred if at least one tumor cell line contained in the mix maintains a cell viability higher than 80% after heat shock (before lysed), with low presence of necrotic and/or apoptotic cell evidence (FIG. 3).

Cells treated in this way are subject to 1 to 6 freezing and thawing cycles, preferably 2 to 4 cycles, and ideally 3 cycles are used. For each freezing cycle, the cells are introduced into a tank containing liquid nitrogen, which freezes them instantly and then thawed to 35 to 40° C.

The lysate or extract obtained is subject to homogenization by ultrasound for 30-second 2 to 10 cycles at 30 to 40 KHz in a standard sonicator. Finally, the lysate or extract of each tissue is irradiated at doses ranging between 40 and 120 Gy, preferably between 70 and 90 Gy and preferentially around 80 Gy. Later, the lysate may be mixed or not on equal parts or individually used depending on the type of tumor to be treated. The lysate or extract obtained is used in the culture of DCs at a concentration between 1 μg/ml and 1 mg/ml and ideally around 100 μg/ml.

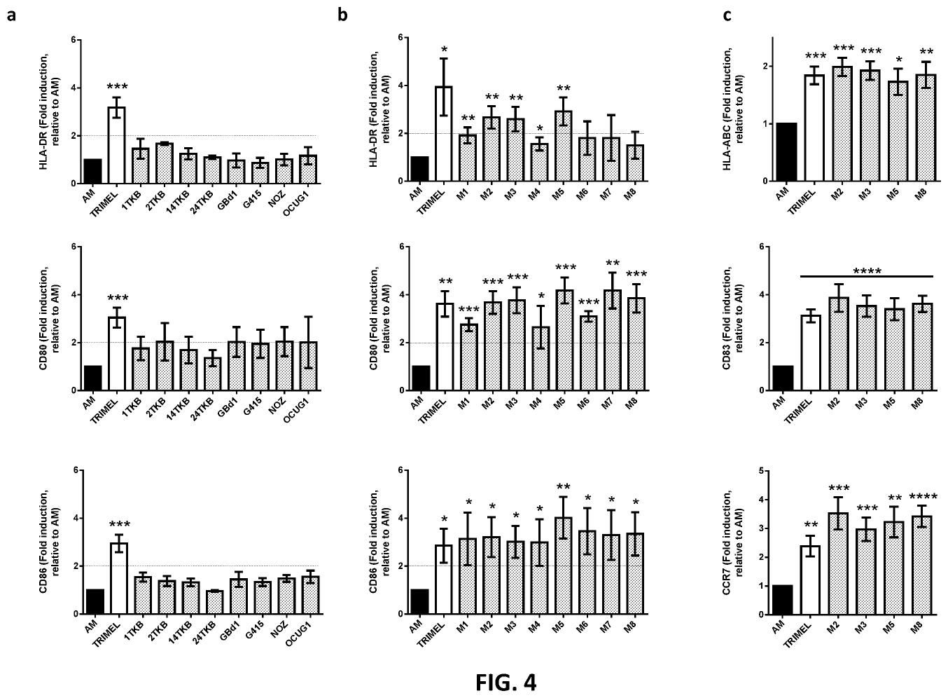

A quite outstanding development of this invention is that the extract of tumor cell lysate described is able to stimulate the differentiation of DCs from preactivated monocytes with differentiation cytokines. This maturation induction and differentiation occurs even in the absence of other cytokines or maturation factors existing in the state of the art. In these cases, it was noted that after hours of treatment with the lysate, monocytes showed a morphology equivalent to DCs classically incubated for 7 days (FIG. 3), which confirms the advantages of the method proposed and the prominent qualities of the extract developed. Also, the monocytes activated with tumor cells extracts showed the CD11c membrane marker expression, which is characteristic of the myeloid-type DCs in addition to the expression of a number of membrane markers characteristic of mature DCs, such as MHC I and MHC II, CD83, CD86, CD40 and CCR7 (FIGS. 4 to 6).

Accordingly, in one aspect the invention provides lysates from a mix of two or more CTCLs that at least one CTCL maintain a cell viability higher than 80% after heat shock, with low presence of necrotic and/or apoptotic cell evidence and detectable levels of DAMPs.

Expressed in another way, the invention provides an immunogenic formulation capable of improving DC capacity to cross-present TAAs for treating cancer in a subject.

Moreover, the invention provides an immunogenic formulation capable to improve CD3⁺ and CD8⁺ T cell infiltration of tumors inhibiting tumor growth in a murine model.

Also provided is the immunogenic formulation comprising an immune stimulant and glycosylated adjuvant including but not limited to mollusk hemocyanin.

In yet another object, the invention provides a method to obtain the immunogenic formulation from lysates from tumor cell lines or from fresh metastatic tissues submitted to the detection of TAAs, heat-shock conditioning and detection of DAMPs. The method further comprises selection, admixing and disruption of selected cell lines, followed by homogenization, irradiation and mixing with adjuvant of the selected lysates.

### EXAMPLES

The invention is further illustrated by the following non-limiting examples.

### Example 1

### Selection of Tumor Cell Lines Based on the Expression of Tumor Associated Antigens

In order to select tumor cell lines suitable for the production of cell lysates as a source of whole tumor antigens, the expression levels of 10 of the most common and relevant tumor associated antigens (Survivin, MUC1, CEA, erbB2, CA19-9, MAGE-1, MAGE-2, MAGE-3, GAGE-1/2 and BAGE) were determined in eight publicly available gallbladder cancer cell lines (GBCCL) (GBd1, G415, OCUG-1, NOZ, 1TKB, 2TKB, 14TKB and 24TKB) and in one GBCCL established in house (CAVE). The protein levels of Survivin, MUC1, CEA, erbB2 and CA19-9 were determined by flow cytometry whereas the expression of MAGEs, GAGEs and BAGE was evaluated at the mRNA level by RT-PCR. The 9 GBCCL showed diverse levels and patterns of antigen expression and none of them expressed all 10 antigens, but all expressed at least two of them (FIG. 1). The expression of erbB2 was detected in all cell lines analyzed, whereas the 2TKB cells only expressed the antigens GAGE1/2 and BAGE. The cell lines with the broader pattern of antigen expression were 2TKB and 1TKB, which express 8 and 7 of the 10 antigens evaluated, respectively (FIG. 1). A similar approach was used to select three melanoma cell lines (Mel1, Mel2, and Mel3), which, in combination express 10 of the most common melanoma associated antigens (MART-1, gp100, tyrosinase, NY-ESO-1, MAGE1, MAGE3, MC1R, MCSP, survivin, and Her2/neu) (Aguilera et al. 2011. Heat-shock induction of tumor-derived danger signals mediates rapid monocyte differentiation into clinically effective dendritic cells. Clin Cancer Res 17:2474-2483).

### Example 2

### Selection of Tumor Cells Based on the Level of Heat Shock Inducible Damage Associated Molecular Patterns (DAMPs)

We evaluated the production of three common DAMPs (released HMGB1 and ATP, and translocated eCRT) in GBCCL and melanoma cells subjected to heat shock. Heat shock treatment induced HMGB1 and ATP release in four of the eight GBCCL evaluated (14TKB, G415, GBd1 and NOZ for ATP; and 2TKB, 24TKB, G415 and OCUG1 for HMGB1) (FIGS. 2a and b). Additionally, three GBCCL translocated eCRT to the plasma membrane in response to heat shock (2TKB, GBd1 and OCUG1) (FIG. 2 c). The levels of heat shock-induced DAMPs in GBCCL were similar that those induced in the melanoma cell lines Mel1, Mel2 and Mel3, which were used as positive controls.

### Example 3

### Heat Shock Treatment of a Mix of Three Melanoma Cell Lines without Impairment of Cell Viability

The method of the present invention for heat shock conditioning of tumor cell lines differs from others in that it does not induce significant levels of cell death, indicating that the heat shock-induced DAMPs could be generated by live cells. Here, after heat shock conditioning of a Mel1+Mel2+Mel3 mix (TRIMEL composition), 80% of the cells remains alive, whereas less than 50% of cell viability was observed when the cells were subjected to a more aggressive heat shock treatment or when they are killed by three cycles of freeze and thaw (FIGS. 3 A, B). Similar results were obtained when 8 different GBCCLs were treated by our heat shock regimen (FIG. 3C).

### Example 4

### Selection of Heat Shock-Conditioned Tumor Cell Lysate Mixtures, Based on the Induction of Differentiation of Activated Monocytes into Mature DCs

We elaborated eight different (M1-M8) heat shock-conditioned lysates combining three different GBCCLs in each lysate. The cell lines composing each mixture lysate were chosen according to their tumor antigen expression and presence of heat shock-induced DAMPs. Unlike individual cell lysates, GBCCL mixture lysates significantly induced the expression of CD80, CD86 and HLA-DR in DCs (FIG. 4a). We extended the analysis to three additional markers: HLA-ABC, CD83 and CCR7 for 4 of these mixtures of GBCCL lysates: M2, M3, M5 and M8 (FIG. 4b), which were selected considering the antigen expression, heat shock-induced DAMP production of the composing cells, and the DC stimulatory activity of the lysate. The addition of M2, M3, M5, M8 or TRIMEL lysate as a control mediated the induction of these maturation markers in DCs (FIG. 4b). As comparison, the addition of TRIMEL to IL-4/GM-CSF-activated monocytes (AM) mediated up to 3-fold induction of surface markers associated with DC maturation such as HLA-DR, CD80 and CD86 (FIG. 4a), however, heat shock-conditioned lysates prepared from each of the GBCCL did not induce a significant increase in the expression of these markers in stimulated AM (FIG. 4c).

### Example 5

### Recognition of HLA-A2-Matched GBC Cell Lines by T Cells Activated by Autologous Monocyte-Derived DCs Loaded with a Heat Shock Conditioned GBC Lysate

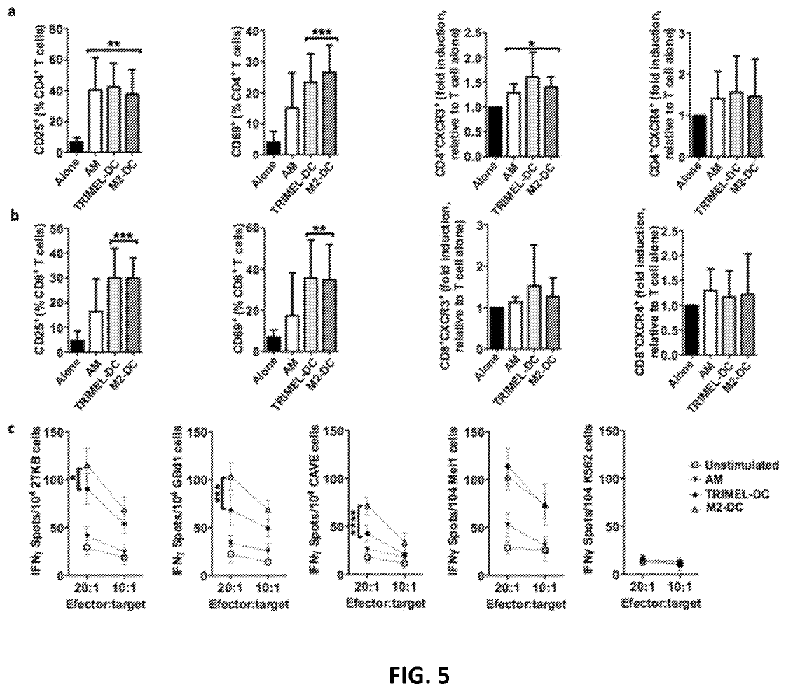

We investigated whether CD8⁺ tumor-specific IFN-γ-secreting T cells were also being elicited in vitro by autologous HLA-A2⁺M2-DCs. First, we observed that M2-DCs were able to activate autologous CD4⁺ and CD8⁺ T cells, measured by the percentage of T cells expressing CD25 and CD69 after 14 days of co-culture (FIGS. 5a and b). Then, CD8⁺ T cells were isolated after co-culture by cell-sorting and challenged with two HLA-A2⁺ GBCCL present in the M2 lysate (2TKB and GBd1), a HLA-A2⁺ GBCCL that was not included in the M2 lysate (CAVE), a HLA-A2⁺ melanoma cell line (Mel1), or with K562 cells (HLA⁻) as a negative control. M2-DC-activated CD8⁺ T cells released significantly higher levels of IFN-γ than CD8⁺ T cells unstimulated or co-cultured with AM or TRIMEL-DCs after being challenged with 2TKB, GBd1 or CAVE cells (FIG. 5c). The NK cell-sensitive cell line K562 did not induce IFN-γ release by the activated CD8⁺ T cells. Additionally, we observed that there was an important cross-recognition of melanoma cells by T cells activated with M2-DCs (FIG. 5c). Similarly, T cells activated with TRIMEL-DCs were able to cross-recognize GBC cells, which may be indicative of shared antigens between both kinds of tumor cells.

### Example 6

### Induction of Murine DC Maturation in Vitro by Heat Shock Conditioned Human Melanoma Cell Lysate (TRIMEL)

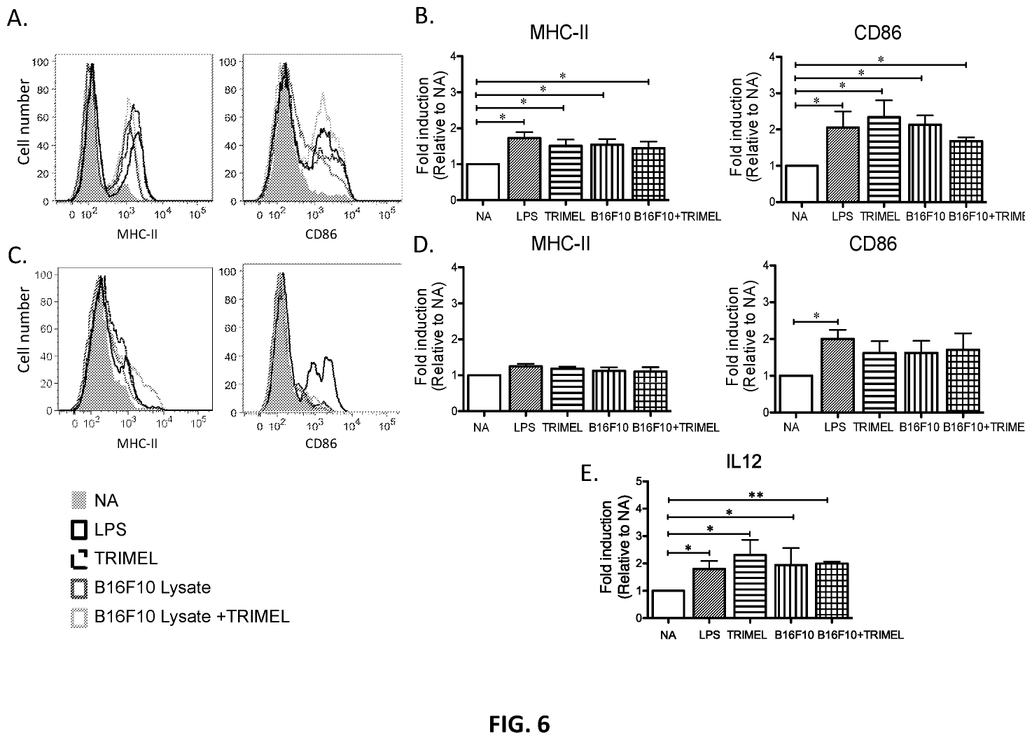

We first tested whether TRIMEL and heat shock conditioned lysate of murine melanoma B16F10 cells induce the activation of murine DCs. Splenic DCs isolated from C56BL/6 mice were stimulated in vitro with TRIMEL, heat shock conditioned lysate from B16F10 cells or a mix of both and checked for the level of expression of MHC-II or CD86 in the surface of conventional DCs (cDCs) and plasmocytoid DCs (pDCs). Both lysates were able to induce an increase in the expression of both maturation markers on cDCs both not in pDCs (FIGS. 6a-d). Moreover, DCs stimulated with these lysates (from human or murine melanoma cells) secreted higher levels of IL-12 than unstimulated DCs (FIG. 6e). These results suggested that murine DCs can sense tumor lysates from human origin and encouraged us to further analyse the potential antitumor activity of heat shock conditioned tumor cell lysate vaccine in a murine model of melanoma.

### Example 7

### B16F10 Tumor Growth is Inhibited by Treatment with LCVX

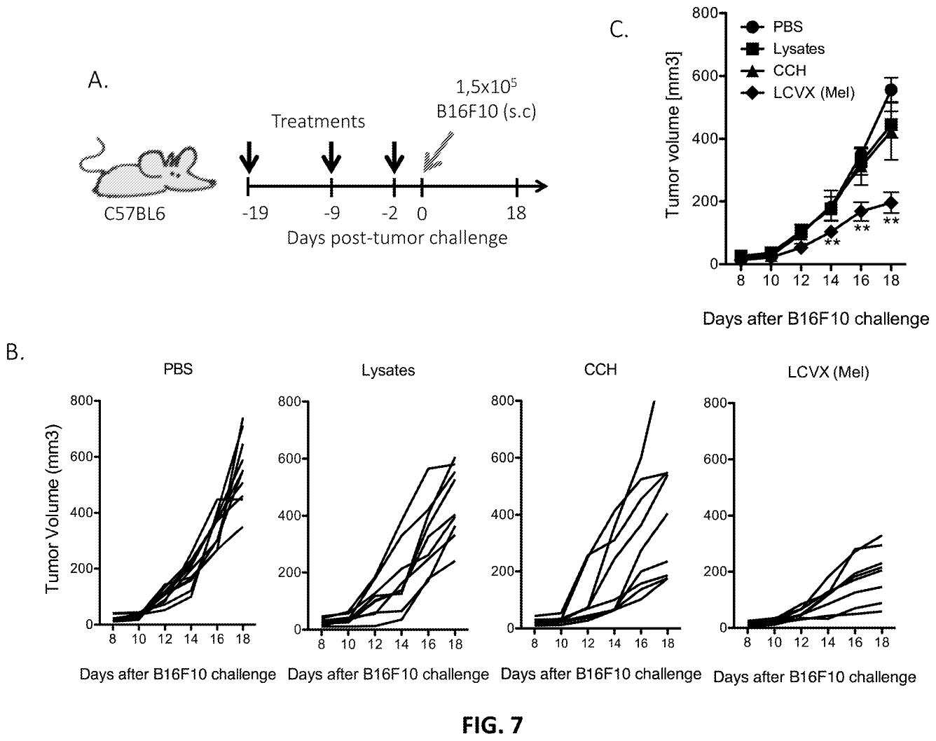

We developed an in vivo model to test the antitumor activity of a vaccine based on TRIMEL against B16F10 melanoma tumors implanted in B57BL/6 mice. As a first approach, we performed a prophylactic setting of vaccination, where C57BL/6 mice were vaccinated three times with: i) LCVX (TRIMEL+B16F10 heat conditioned lysate+CCH); ii) Lysates alone (TRIMEL+B16F10 heat conditioned lysate); iii) CCH alone, or iv) PBS (vehicle). Then, the mice were challenged with B16F10 melanoma cells, and tumor growth was monitored for 18 days after challenging (FIG. 7a). The complete composition of LCVX, in this murine model, must contain TRIMEL (as source of tumor-associated DAMPs), B16F10 lysate (as source of murine melanoma associated antigens), and CCH as a potent adjuvant. As observed in FIG. 7b-c, LCVX treatments induced a potent tumor growth retardation in this prophylactic setting, while each vaccine component alone did not.

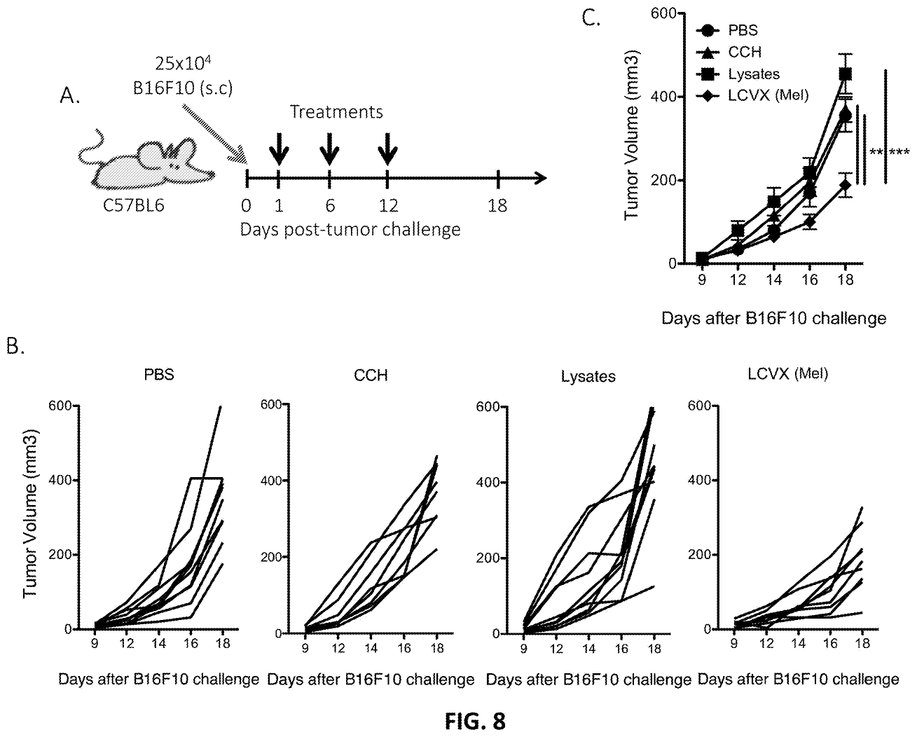

The same protective antitumor response was observed in tumor bearing mice in a therapeutic approach (FIG. 8). Mice were challenged with B16F10 melanoma cells, and 1 day post-tumor challenging were vaccinated three times with: i) LCVX (TRIMEL+B16F10 heat conditioned lysate+CCH); ii) Lysates alone (TRIMEL+B16F10 heat conditioned lysate); iii) CCH alone, or iv) PBS (vehicle). Tumor growth was monitored for 19 days after challenging (FIG. 8a). As observed in FIGS. 8b-c, therapeutic LCVX treatments induced a potent tumor growth retardation, while each vaccine component alone did not.

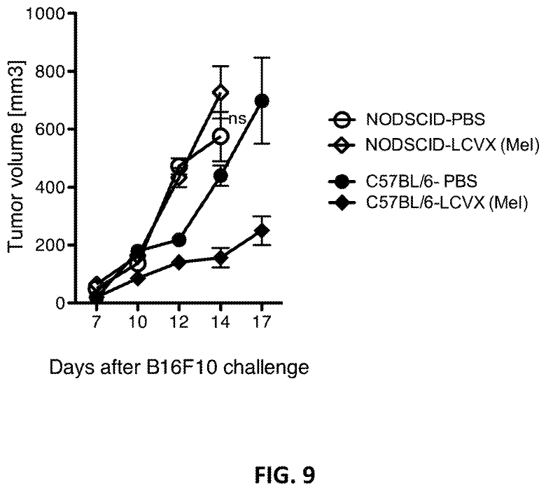

In other set of experiments, C57BL/6 or immunodeficient NODSCID mice were vaccinated three times with: i) LCVX (TRIMEL+B16F10 heat conditioned lysate+CCH); or ii) PBS (vehicle). Then, the mice were challenged with B16F10 melanoma cells, and tumor growth was monitored for 17 days after challenging (as described for FIG. 7a). The results demonstrated that such LCVX antitumor activity depends of a competent immune system, as it fails in induce tumor growth inhibition in immunodeficient NODSCID mice bearing B16F10 tumors (FIG. 9).

### Example 8

### Evaluation of Lysate Concentration, B16F10-Derived Antigens and Different Hemocyanin Adjuvants in the Tumor Protective Activity of LCVX

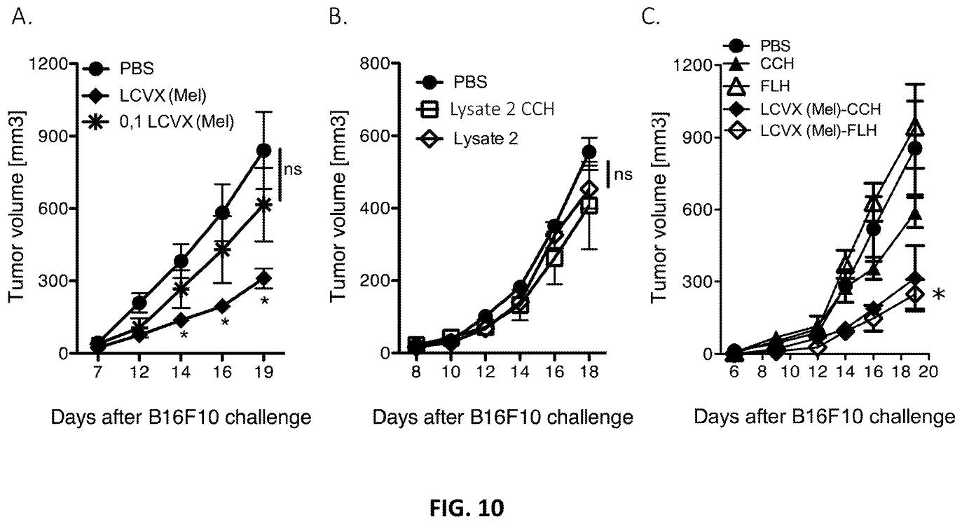

Additionally, we determined the dose-dependent effect of the TRIMEL lysate on LCVX antitumor activity. C57BL/6 female mice were inoculated s.c. at days −19, −9 and −2 (before tumor challenging) with two different doses of LCVX: i) 1 mg of lysate protein/dose/animal (LCVX), or ii) 0.1 mg of lysate protein/dose/animal (LCVX (1/10)), or iii) vehicle (PBS). At the day 0, mice were challenged (s.c.) with B16F10 cells and tumor growth was monitored every 2 days for 19 days post-tumor challenging. The results showed that by decreasing the TRIMEL concentration 10 times the tumor protective activity of LCVX was lost (FIG. 10a), suggesting that 1 mg of lysate protein/dose contained the optimal DAMP and TAAs concentrations for potent antitumor activity in vivo.

In other experiments using the same prophylactic setting, mice were vaccinated with: i) TRIMEL lysate alone, ii) TRIMEL+CCH, or iii) PBS. The results indicated that the presence of B16F10-derived antigens is fundamental for LCVX-mediated tumor growth inhibition, and that the human melanoma associated antigens present in the TRIMEL lysate do not cross-react with B16F10 antigens (FIG. 10b).

Additionally, we also compared the effectivity of two different hemocyanin derived adjuvants (CCH and FLH) in the activity of LCVX (using the same prophylactic setting as before). Our results suggest that both adjuvants were equally effective in inducing antitumor immunity in combination with heat shock conditioned tumor lysates (FIG. 10c).

### Example 9

### Heat Shock-Conditioned Gallbladder Cancer Cell Lysate Vaccine LCVX (GBC) Promotes B16F10 Tumor Growth Inhibition

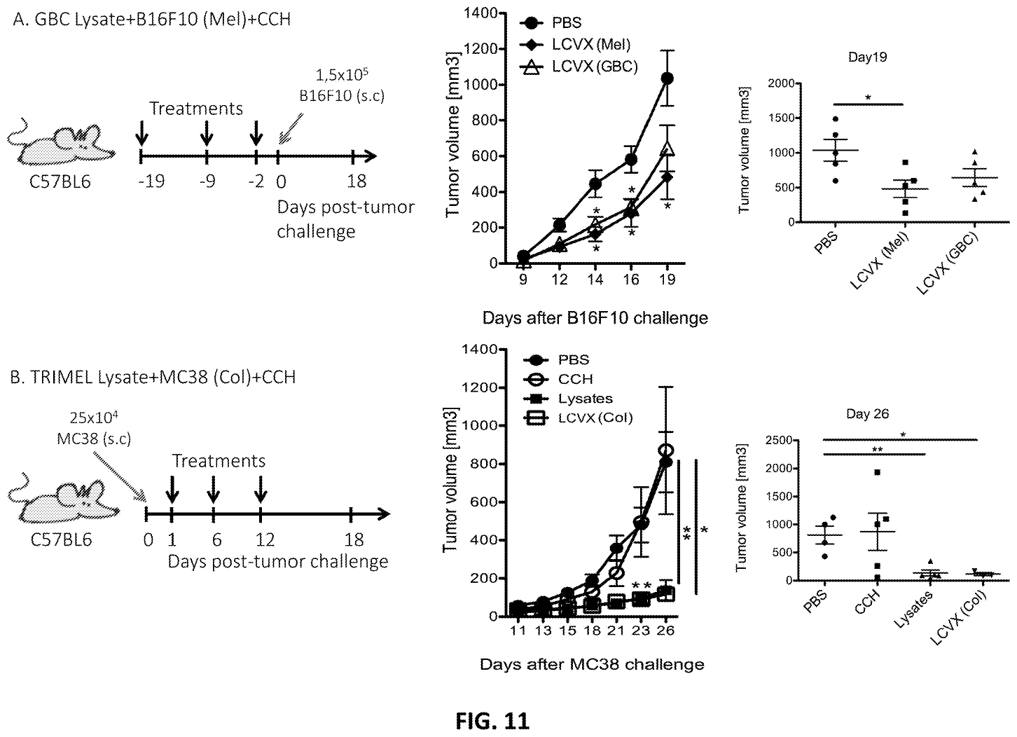

C57BL/6 female mice were challenged s.c. at day 0 with 0.25×10⁵ B16F10 cells and then inoculated s.c. at days 1, 6 and 12 post-tumor challenging with: i) LCVX (Mel) (TRIMEL+B16F10 HS lysate+CCH), ii) LCVX (GBC) (M2 GBC cell lysate+B16F10 HS lysate+CCH), or iii) vehicle (PBS) and the tumor growth was monitored every 2 days for 19 days post-tumor challenging. The results suggest that the lysate of three GBCCLs (M2), when combined with B16F10 HS lysate and CCH, induce a potent tumor growth inhibition as TRIMEL does (FIG. 11A). These observations suggest that the method to generate HS conditioned human tumor cell lysates produce effective levels of DAMPs that can protect against cancer in vivo in models of different tumor origin. Additionally, LCVX vaccine can be effective to inhibit different kinds of murine tumors such as a colon adenocarcinoma MC38. C57BL/6 female mice were challenged s.c. at day 0 with 0.25×10⁵ MC38 cells and then inoculated s.c. at days 1, 6 and 12 post-tumor challenging with: i) LCVX (Col) (TRIMEL +MC38 HS lysate +CCH), ii) Lysates (TRIMEL+MC38 HS lysate), iii) CCH alone or iv) vehicle (PBS). Tumor growth was monitored every 2 days for 26 days post-tumor challenging. The results indicated that the presence of MC38-derived antigens combined with TRIMEL lysate also is effective for LCVX-mediated tumor growth inhibition, even in the absence of the CCH adjuvant (FIG. 11B) suggesting the potential use of LCVX in the treatment of different types of cancer.

### Example 10

### The Anti-Tumor Effect of the Anti-PD-1 Immune Checkpoint Inhibitor is Improved by Combination with LCVX Therapy

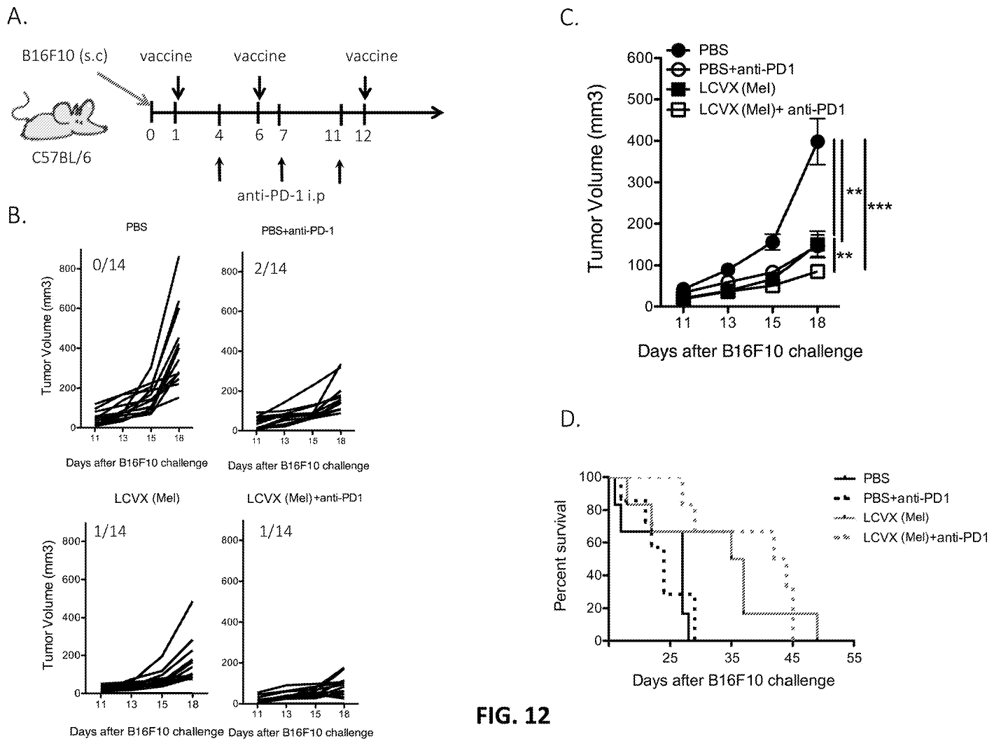

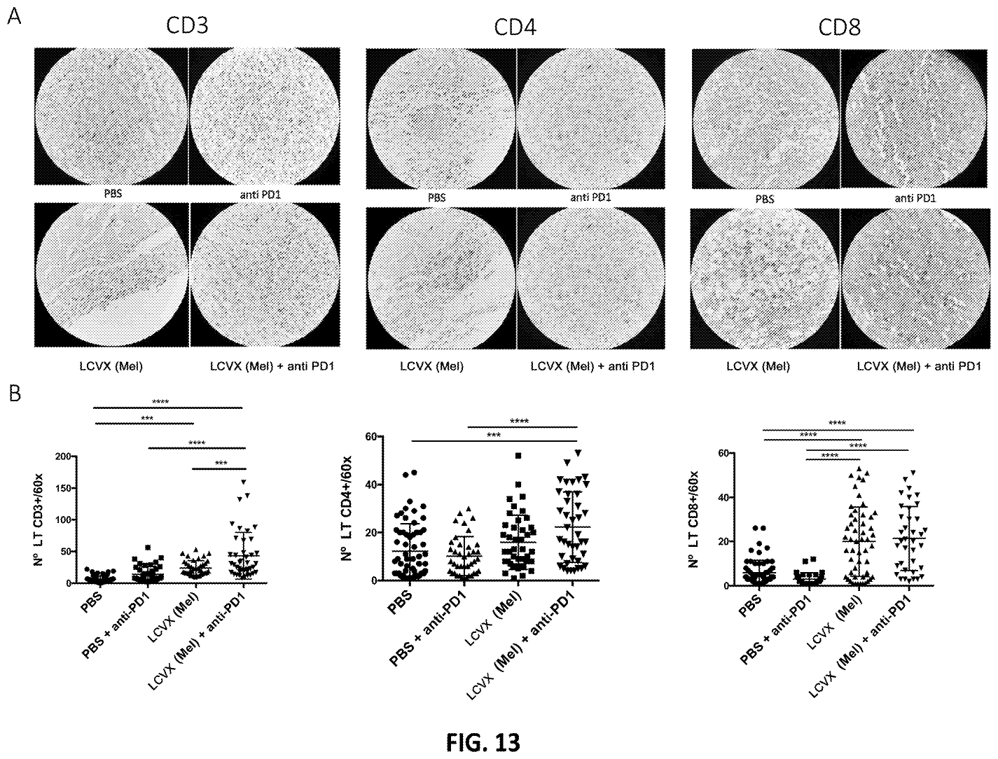

C57BL/6 female mice were challenged s.c. at day 0 with 0.25×10⁵ B16F10 cells and then inoculated s.c. at days 1, 6 and 12 post-tumor challenging with: LCVX (TRIMEL+B16F10 HS lysate+CCH), or vehicle (PBS). Additionally, mice received three i.p. doses of anti-PD-1 antibodies (or vehicle PBS) at days 4, 7 and 11 post-tumor challenging. The tumor growth and survival of mice were monitored every 2 days for 18 or 35 days post-tumor challenging, respectively (FIG. 12a). Our results showed that both therapeutic LCVX or anti-PD-1 inhibitor treatments induced potent tumor growth retardation, and the combination of both therapies leads to a more potent antitumor effect, determined by tumor growth retardation or increased post-tumor challenging survival (FIGS. 12b-d). In an additional experiment, mice treated as described above were sacrificed at day 15 post tumor challenge and the obtained tumors were analyzed for the infiltration of CD3⁺, CD4⁺ and CD8⁺ T cells. LCVX treated mice showed increased infiltration of CD3⁺ and CD8⁺ T cells compared to control mice or mice treated only with anti-PD-1, indicating an increasing in cytotoxic potential against tumors. Moreover, LCVX treatment enhanced CD3⁺, CD4⁺ and CD8⁺ T cell infiltration to tumors when combined with anti-PD-1 treatment (FIGS. 13a-b) in line with the enhanced antitumor effect observed for combined therapy.

The invention should be understood as broad as the disclosed preferred embodiments, its optional features and the prior art permit. Any modification and variation of the concepts herein disclosed that will be apparent to those of skilled in the art, whether now existing or later developed, are deemed to be within scope and spirit of the invention as defined above and by the appended claims.

What is claimed is:

1. An immunogenic formulation for treating a cancer in a subject comprising:
i) an immunologically effective amount of two or more cell lysates generated from heat shock-conditioned cancer cell populations, wherein each heat shock-conditioned cancer cell population immediately before lysis (a) expressed two or more tumor-associated antigens (TAAs), (b) had elevated levels of two or more damage-associated molecular pattern molecules (DAMPs), and (c) had a cell viability of higher than 80%; and
ii) an immunologically effective amount of an adjuvant.

2. The immunogenic formulation of claim 1, wherein the cell viability is assessed by the absence of necrotic or apoptotic signals.

3. The immunogenic formulation of claim 1 or claim 2, wherein the combined two or more cell lysates comprise at least three TAAs and at least three DAMPs at elevated levels.

4. The immunogenic formulation of any one of claims 1-3, wherein the adjuvant is selected from the group consisting of glycosylated adjuvant, a carrier adjuvant, a Very Small Size Proteoliposome adjuvant (VSSP), an oil-in-water emulsion, a saponin-based adjuvant, a mineral salt adjuvant, an immunostimulant, and any combinations thereof.

5. The immunogenic formulation of any one of claims 1-3, wherein the adjuvant comprises a glycosylated adjuvant.

6. The immunogenic formulation of claim 5, wherein the glycosylated adjuvant is a particular hemocyanin or combinations of particular hemocyanins.

7. The immunogenic formulation of claim 6, wherein the particular hemocyanin is obtained from mollusk, preferably species from Muricidae, Fissurellidae and Haliotidae families.

8. The immunogenic formulation of claim 6, wherein the particular hemocyanin is Keyhole limpet hemocyanin (KLH), Concholepas concholepas hemocyanin (CCH), or Fissurella latimarginata hemocyanin (FLH).

9. The immunogenic formulation of any one of claims 6-8, wherein the immunogenic formulation comprises at least 0.5 micrograms of the particular hemocyanin per dose.

10. The immunogenic formulation of any one of claims 6-8, wherein the immunogenic formulation comprises from 0.5 micrograms to 500 micrograms, optionally from 5 micrograms to 150 micrograms, or optionally about 150 micrograms, of the particular hemocyanin per dose.

11. The immunogenic formulation of any one of claims 1-3, wherein the adjuvant comprises a carrier adjuvant, optionally a liposome or a virosome.

12. The immunogenic formulation of any one of claims 1-3, wherein the adjuvant comprises a liposome and the immunogenic formulation comprises from 0.5 micrograms to 200 micrograms of the liposome per dose.

13. The immunogenic formulation of any one of claims 1-3, wherein the adjuvant comprises a virosome and the immunogenic formulation comprises from 0.1 micrograms to 5 mg of viral protein of the virosome per dose.

14. The immunogenic formulation of any one of claims 1-3, wherein the adjuvant comprises a VSSP, optionally a ganglioside M3 (GM3), and optionally the immunogenic formulation comprises from 10 micrograms to 300 micrograms of GM3 per dose.

15. The immunogenic formulation of any one of claims 1-3, wherein the adjuvant comprises an oil-in-water adjuvant, optionally MF59 or montanide.

16. The immunogenic formulation of any one of claims 1-3, wherein the adjuvant comprises MF59 and the immunogenic formulation comprises from 0.2% to 20% (vol/vol) of MF59.

17. The immunogenic formulation of any one of claims 1-3, wherein the adjuvant comprises montanide and the immunogenic formulation comprises from 2% to 70% (vol/vol) of montanide.

18. The immunogenic formulation of any one of claims 1-3, wherein the adjuvant comprises a saponin-based adjuvant, optionally immunostimulatory complexes (ISCOMs) or Quillaja saponaria-21 (QS-21).

19. The immunogenic formulation of any one of claims 1-3, wherein the adjuvant comprises ISCOMs and the immunogenic formulation comprises from 0.5 micrograms to 50 micrograms of ISCOMs per dose.

20. The immunogenic formulation of any one of claims 1-3, wherein the adjuvant comprises QS-21 and the immunogenic formulation comprises from 0.01 micrograms to 30 micrograms of QS-21 per dose.

21. The immunogenic formulation of any one of claims 1-3, wherein the adjuvant comprises a mineral salt adjuvant, optionally alum, aluminum salt and TLR4 agonist-based adjuvant, optionally AS01, AS02, AS03, AS04, or AS15.

22. The immunogenic formulation of any one of claims 1-3, wherein the adjuvant comprises alum or an aluminum salt and the immunogenic formulation comprises from 1 micrograms to 50 mg of alum or the aluminum salt per dose.

23. The immunogenic formulation of any one of claims 1-3, wherein the adjuvant comprises TLR4 agonist-based adjuvant, optionally AS01, AS02, AS03, AS04, or AS15, and the immunogenic formulation comprises from 0.1 micrograms to 20 micrograms of TLR4 agonist-based adjuvant, optionally AS01, AS02, AS03, AS04, or AS15, per dose.

24. The immunogenic formulation of any one of claims 1-3, wherein the adjuvant comprises an immunostimulant, optionally a Toll like receptor (TLR) ligands (optionally, Poly I:C, poly-ICLC, monophosphoryl lipid A (MPL), glucopyranosyl lipid adjuvant (GLA), imiquimod, or CpG ODN) or polysaccharides (optionally, chitin, chitosan, or β-glucan).

25. The immunogenic formulation of any one of claims 1-3, wherein the adjuvant comprises Poly I:C or poly-ICLC and the immunogenic formulation comprises from 0.1 mg to 10 mg of Poly I:C or poly-ICLC per dose.

26. The immunogenic formulation of any one of claims 1-3, wherein the adjuvant comprises MPL and the immunogenic formulation comprises from 5 micrograms to 500 micrograms of MPL per dose.

27. The immunogenic formulation of any one of claims 1-3, wherein the adjuvant comprises GLA and the immunogenic formulation comprises from 0.5 micrograms to 50 micrograms of GLA per dose.

28. The immunogenic formulation of any one of claims 1-3, wherein the adjuvant comprises imiquimod and the immunogenic formulation comprises from 25 mg to 500 mg of imiquimod per dose.

29. The immunogenic formulation of any one of claims 1-3, wherein the adjuvant comprises CpG ODN and the immunogenic formulation comprises from 50 micrograms to 10 mg of CpG ODN per dose.

30. The immunogenic formulation of any one of claims 1-3, wherein the adjuvant comprises chitin or chitosan and the immunogenic formulation comprises from 0.01 mg to 100 mg of chitin or chitosan per dose.

31. The immunogenic formulation of any one of claims 1-3, wherein the adjuvant comprises β-glucan and the immunogenic formulation comprises from 0.1 mg to 500 mg of β-glucan per dose.

32. The immunogenic formulation of any one of claims 1-31, wherein the two or more DAMPs are selected from the group consisting of post-heat shock and pre-lysis secretion of: chromatin-associated protein high-mobility group box 1 protein (HMGB1), ATP, S100/Calgranulin protein family members [optionally, S100 calcium binding protein A8 (S100A8), S100 calcium binding protein A9 (S100A9), and/or S100A12/EN-RAGE], Heat shock protein (HSP) 70 (HSP70), HSP90, HSP60, HSP72, nucleic acids (optionally mitochondrial DNA, dsDNA, and dsRNA), Prostaglandin E2 (PGE2), Monosodium urate (MSU), uric acid, and Peroxiredoxin 1 (Prx1), pre-lysis plasma membrane expression of: Calreticulin (CRT) and Death domain 1 alpha (DD1alpha); and elevated levels of HSP70, HSP72, HSP60, and HSP72.

33. The immunogenic formulation of any one of claims 3-32, wherein the at least three DAMPs include one, two, or all three of post-heat shock and pre-lysis secretion of HMGB1 and/or lysis plasma membrane expression of CRT.

34. The immunogenic formulation of any one of claims 3-33, wherein the at least three DAMPs are in an effective amount to induce activation and maturation of antigen-presenting cells (APCs) when administered to a subject.

35. The immunogenic formulation of any one of claims 1-34, wherein the two or more TAAs are selected from the group consisting of melanoma antigen recognized by T cells 1 (MART1), glycoprotein 100 (gp100), tyrosinase, New York esophageal squamous cell carcinoma 1 (NY-ESO-1), melanoma-associated antigen 1 (MAGE1), melanoma-associated antigen 2 (MAGE2), melanoma-associated antigen 3 (MAGE3), melanocortin 1 receptor (MC1R), melanoma-associated chondroitin sulfate proteoglycan (MCSP), survivin, human epidermal growth factor receptor (Her2), carbohydrate antigen (CA) 19-9 (CA10-9), mucin 1 (MUC1), mucin 5AC (MUC5AC), carcinoembryonic antigen (CEA), G antigen 1 (GAGE1), G antigen 2 (GAGE2), B melanoma antigen (BAGE), cytokeratin 7 (CK7), Cytokeratin 19 (CK19), and cancer antigen 125 (CA125).

36. The immunogenic formulation of any one of claims 1-35, wherein the two or more cell lysates are in an amount effective to induce the release of two or more proinflammatory cytokines selected from the list consisting of IL-6, IL-8, TNF-α, IL-10, IL-1, IFN-γ, and IL-12.

37. The immunogenic formulation of any one of claims 1-35, wherein the two or more cell lysates are in an amount effective to induce the release of TNF-α and IL-12.

38. The immunogenic formulation of any one of claims 1-37, wherein the two or more cell lysates are in an amount effective to induce the overexpression of three or more maturation-associated markers on antigen-presenting cell membrane selected from the group consisting of MHC class I, MHC class II, CD83, CD86, CD80, CD40, CCR7, DEC-205, DC-SIGN and MICA.

39. The immunogenic formulation of any one of claims 1-37, wherein the two or more cell lysates are in an amount effective to induce the overexpression of CD83, CD86, and CD80.

40. The immunogenic formulation of any one of claims 1-39, wherein the two or more cell lysates are in an amount effective to improve dendritic cells (DCs) capacity to cross-present TAAs.

41. The immunogenic formulation of any one of claims 1-40, wherein the immunogenic formulation comprises each cell lysate was produced from at least 50,000 cells per dose.

42. The immunogenic formulation of any one of claims 1-41, wherein the immunogenic formulation comprises a total cell lysate produced from 100,000 to 50,000,000 cells per dose, optionally about 5,000,000 cells per dose.

43. The immunogenic formulation of any one of claims 1-42, wherein at least one of the two or more cell lysates is generated from cancer cell lines selected from the group consisting of malignant melanoma, prostate cancer, gallbladder cancer, lung cancer, breast cancer, colon cancer, kidney cancer, kidney cancer, cervical cancer, ovarian cancer, gastric cancer, brain cancer, and pancreatic cancer.

44. The immunogenic formulation of any one of claims 1-43, wherein at least one of the two or more cell lysates is generated from fresh metastatic tumor tissues.

45. The immunogenic formulation of claim 44, wherein the fresh metastatic tumor tissue is obtained from the subject to be treated.

46. The immunogenic formulation of any one of claims 1-45, wherein the two or more cell lysates are autologous, allogeneic, or combinations with respect to the subject to be treated.

47. The immunogenic formulation of any one of claims 1-46, wherein the immunogenic formulation is suitable for administration by subcutaneous, intradermal, intratumoral, or intranodal injection.

48. A method of generating the immunogenic formulation of any one of claims 1-47, comprising admixing the two or more cell lysates and the immunologically effective amount of the adjuvant.

49. The method of claim 48, wherein the two or more cell lysates are admixed before addition of the immunologically effective amount of the adjuvant.

50. The method of claim 49, wherein the two or more cell lysates are admixed with the immunologically effective amount of the adjuvant immediately prior to administration to the subject or up to 48 hours after admixture.

51. The method of any one of claims 48-50, wherein the two or more cell lysates and optionally the adjuvant are lyophilized and store prior to use and are reconstituted by admixture with the immunologically effective amount of the adjuvant immediately or with sterile water immediately prior to administration to the subject or up to 48 hours after admixture.

52. The method of any one of claims 48-51, where at least one of the two or more cell lysates is generated by:
i) providing a cancer cell line that expresses the two or more TAAs;
ii) incubating the cancer cell line at a temperature and for a time sufficient to induce heat shock to produce a population of heat-shocked cancer cells;
iii) incubating the population of heat-shocked cancer cells at 37° C. for a time sufficient to induce the elevated levels of the two or more DAMPS to produce the heat shock-conditioned cancer cell population with the cell viability of higher than 80%;
iv) disrupting the heat shock-conditioned cancer cell population to produce at least one cell lysate, optionally by mechanical disruption, sonication, microwave, blenders, standard liquid homogenizers, or high pressure homogenizers; and
v) homogenizing the at least one cell lysate.

53. The method of claim 52, wherein the cancer cell line is provided by obtaining cells from fresh metastatic tumor tissue.

54. The method of claim 53, wherein the fresh metastatic tumor tissue is obtained from the subject.

55. The method of any one of claims 52-54, wherein the cancer cell line is provided by screening one or more cancer cell lines for expression of the two or more TAAs and selecting the cancer cell line that expresses the two or more TAAs.

56. The method of any one of claims 52-55, wherein the temperature sufficient to induce heat shock is between 39° C. and 45° C., optionally at about 42° C.

57. The method of any one of claims 52-56, wherein the time sufficient to induce heat shock is between 15 minutes and 3 hours, optionally about 1 hour.

58. The method of any one of claims 52-57, wherein the cells in step ii) are in a serum-free, red-phenol-free culture medium optionally AIM-V red phenol-free or PBS+human serum albumin (0.1-5%).

59. The method of any one of claims 52-58, wherein the time to induce the elevated levels of the two or more DAMPS is from 0.5 hours to 6 hours, optionally from 1 to 3 hours, and optionally about 2 hours.

60. The method of any one of claims 52-59, wherein after step iii), the heat shock-conditioned cancer cell population is screened for expression of the two or more DAMPs and steps i)-iii) are repeated if the heat shock-conditioned cancer cell population does not express the two or more DAMPs.

61. The method of any one of claims 52-59, wherein after step iv), the at least one cell lysate is screened for presence of the two or more DAMPs and steps i)-iv) are repeated if the at least one cell lysate does not comprise the two or more DAMPs.

62. The method of any one of claims 52-61, wherein the heat shock-conditioned cancer cell population are admixed with one or more additional heat shock-conditioned cancer cell populations that (a) express two or more TAAs, (b) have two or more DAMPs, and (c) have a cell viability of higher than 80% before step iv).

63. The method of any one of claims 52-62, where the disrupting comprises at least one cycle of freezing and thawing of the heat shock-conditioned cancer cell population.

64. The method of claim 63, wherein the disrupting comprises 2 to 4 cycles of cycle of freezing and thawing of the heat shock-conditioned cancer cell population.

65. The method of claim 63 or claim 64, wherein the freezing is with liquid nitrogen.

66. The method of any one of claims 63-65, wherein the thawing is at 35-40° C., optionally 37° C.

67. The method of any one of claims 52-66, further comprising a sterilizing step after the incubating step iii).

68. The method of claim 67, wherein the sterilizing step is after the homogenizing step v).

69. The method of claim 67 or claim 68, wherein the sterilizing step comprises irradiation.

70. The method of claim 69, wherein the irradiation comprises a dose from 50 to 100 Gy, optionally 80 Gy.

71. The method of any one of claims 52-70, further comprising testing the at least one cell lysate for inducing the activation of APCs to display a phenotype similar to mature DCs, optionally as tested by cell surface marker expression and cytokine release by flow cytometry.

72. A method of treatment of a cancer in a subject comprising administering the immunogenic formulation of any one of claims 1-47 to the subject.

73. The method of treatment of cancer of claim 72, wherein the cancer is selected from the group consisting of melanoma, malignant melanoma, prostate cancer, gallbladder cancer, lung cancer, breast cancer, colon cancer, kidney cancer, renal cancer, cervix cancer, ovarian cancer, gastric cancer, brain cancer, and pancreatic cancer.

74. The method of treatment claim 72 or claim 73, where (i) and (ii) of the immunogenic formulation are administered separately to the subject at the same time or at different times.

75. The method of treatment any one of claims 72-74, further comprising administering an immune check-point inhibitor agent before, after, or simultaneously with the administration of the immunogenic formulation.

76. The method of treatment of claim 75, wherein the immune check-point inhibitor agent inhibits PD-1, PD-L1 or CTLA4.

77. The method of treatment of claim 75 or claim 76, wherein the immune check-point inhibitor agent is a monoclonal antibody.

78. The method of treatment of claim 77, wherein the monoclonal antibody is selected from the group consisting of pembrolizumab, nivolumab, cemiplimab, atezolizumab, avelumab, durvalumab, ipilimumab, or a biosimilar thereof.
